(19) United States
(12) Patent Application Publication
(10) Pub. No.: US 2011/0165611 A1
(43) Pub. Date: Jul. 7, 2011

(54) DUAL MODALITY DETECTION OF APOPTOSIS

(76) Inventors: Chun Li, Missouri City, TX (US); Chiyi Xiong, Cypress, TX (US)

(21) Appl. No.: 13/062,212

(22) PCT Filed: Sep. 3, 2009
(86) PCT No.: PCT/US09/55899

§ 371 Date: Mar. 3, 2011

Related U.S. Application Data
(60) Provisional application No. 61/094,205, filed on Sep. 4, 2008.

(51) Int. Cl. C12Q 1/02; C12N 9/96

(52) U.S. Cl. 435/29; 435/188

(57) ABSTRACT

To image apoptosis in vivo, small, membrane-permeable probes comprising a caspase 3 substrate, a fluorogenic dye and a radionuclide is provided. This dual-modality probe can be cleaved by caspase upon exposure to apoptotic cells, allowing imaging of caspase 3 and 7 activities using both optical and nuclear imaging techniques. The combined use of these methods provides the opportunity for a direct correlation between in vitro and in vivo biological activities and a viable method to treat disease

### CROSS-REFERENCE TO RELATED APPLICATIONS

This application claims priority to U.S. Pat. App. Ser. No. 61/094,205 which is incorporated by reference herein in its entirety.

### STATEMENT REGARDING FEDERALLY SPONSORED RESEARCH OR DEVELOPMENT

None.

### THE NAMES OF THE PARTIES TO A JOINT RESEARCH AGREEMENT

None.

### INCORPORATION-BY-REFERENCE OF MATERIAL SUBMITTED ON A COMPACT DISC

None.

### BACKGROUND OF THE INVENTION

Fluorogenic DEVD conjugates which become fluorescent in the presence of caspase 3 have been used for in vitro detection of apoptosis in very limited applications. For example, certain fluorogenic DEVD conjugates have been used in fluorescence microscopy, a method used in biomedical research to gain information at the cellular and subcellular level. H.-Z. Zhang, S. Kasibhatla, J. Guastella, B. Tseng, J. Drewe, S. X. Cai, Bioconjug. Chem. 2003, 14, 458-463; B. W. Lee, G. L. Johnson, S. A. Hed, Z. Darzynkiewicz, J. W. Talhouk, S. Mehrotra, Biotechniques 2003, 35, 1080-1085; J. Liu, M. Bhalgat, C. Zhang, Z. Diwu, B. Hoyland, D. H. Klaubert, Bioorg. Med. Chem. Lett. 1999, 9, 3231-3236. However, because of the strong attenuation of fluorescent light and limited penetration depth, use of such conjugates has not been shown viable in vivo to detect apoptosis or in optical imaging in the clinic.

### SUMMARY OF THE INVENTION

Multimodality imaging probes are provided herein. Such probes are capable of detecting the activity of caspases in nuclear and optical imaging applications. These probes can have a fluorescent or fluorogenic compound and a radionuclide or radioactive tag that allow both optical and nuclear imaging. As such, these probes validate in vivo imaging findings. The probe can be cell permeable and, intact, freely diffuses into and out of viable cells. The probe is efficiently cleaved by caspase 3 in apoptotic cells where the cleaved products (including radionuclide and fluorophores) can be trapped within apoptotic cells.

Further provided are novel, dual labeled DEVD peptides, and fluorogenic DEVD conjugates and methods of using the same. Such compositions of matter may be useful in imaging therapy-induced apoptosis in cancer patients as well as apoptotic process in other diseases including heart disease, ischemia, arthrosclerosis, stroke, arthritis and the like.

### BRIEF DESCRIPTION OF THE DRAWINGS

FIG. 1 is an analytical HPLC chromatograms of purified compound S1.

FIG. 2 is an analytical HPLC chromatograms of purified compound S2.

FIG. 3 is an analytical HPLC chromatograms of purified compound 2.

FIG. 4 shows analytical HPLC chromatograms of purified 3.

FIGS. 5 & 6 represent a comparison of chromotograms between Re-chelate 3 and ^{99m}Tc-chelate obtained under the same chromatographic conditions. Re-chlate was detected with an UV detector, ^{99m}Tc-chelate was detected with radiodetector

FIG. 7 is a confocal fluorescence image of apoptotic cells stained with both annexin V (red) and activated dual modality imaging probe (green).

FIGS. 8 A, B, C & D provide fluorescence images acquired with (8A) untreated DLD1 cells, (8B) TRAIL-treated DLD1 cells, and (8C) TRAIL-plus-Ac-DEVD-CHO-treated DLD1 cells. Activation of fluorescent signal resulting from R110-D-SAAC-Re(CO)₃ fragment cleaved from 3 (green) was seen only in TRAIL-treated cells. Annexin V-Alexa Fluor 594-labeled apoptotic cells were pseudocolored red. Note that cells treated with both TRAIL and Ac-DEVD-CHO were stained with annexin V but not with 3. Bar: 200 μm. (8D) Z-stacking of apoptotic cells stained with both annexin V and 3. Fluorescent signal from cleaved 3 was seen in the cytoplasm of cells with intact membrane (arrows). Early stage apoptotic cells stained only with cleaved 3 but not with annexin V was also observed (arrowhead). Bar: 20 μm.

FIG. 9 shows the activation of fluorescent signal in DLD1 cells by TRAIL. The DLD1 cells were sequentially treated with 100 μL of the following agents at 37° C.: (A) 25 μM 2 or 3 for 2 h; (B) 25 μM each compound for 2 h followed by TRAIL (150 ng/mL) for 2 h; (C) 25 μM each compound for 2 h, drug-free culture medium for 2 h, and TRAIL (150 ng/mL) for 2 h; or (D) 25 μM each compound for 2 h, drug-free culture medium for 24 h, and TRAIL (150 ng/mL) for 2 h. Note the reduced fluorescence activity with increasing incubation time in drug-free medium. Also note the activation of 2 and 3 in apoptotic cells in (B); the fluorescence intensities increased by 27- and 99-fold, respectively, upon TRAIL treatment.

FIG. 10 shows the activation of fluorescent signal in DLD1 cells by TRAIL. The DLD1 cells were sequentially treated with 100 μL of the following agents at 37° C.: (A) 25 μM 2 or 3 for 2 h; (B) 25 μM each compound for 2 h followed by TRAIL (150 ng/mL) for 2 h; (C) 25 μM each compound for 2 h, drug-free culture medium for 2 h, and TRAIL (150 ng/mL) for 2 h; or (D) 25 μM each compound for 2 h, drug-free culture medium for 24 h, and TRAIL (150 ng/mL) for 2 h. Note the reduced fluorescence activity with increasing incubation time in drug-free medium. Also note the activation of 2 and 3 in apoptotic cells in (B); the fluorescence intensities increased by 27- and 99-fold, respectively, upon TRAIL treatment.

FIG. 11 depicts a fluorescence microscopic analysis of livers from mice treated with anti-Fas to induce apoptosis (upper panel) and with PBS as a control (lower panel). Tissues were harvested 4 h after drug treatment and 2 h after the intravenous injection of ^{99m}Tc-chelate 4. Activated caspase 3 was stained with anti-caspase 3 antibody (red), and cell nuclei were counterstained with Hoechst 33342 (blue). Signal from cleaved 4 is pseudocolored green. Bar 40 μm

FIGS. 12 A & B shows a comparison of radio-HPLC chromatograms of liver extracts from a PBS-treated mouse (A) and an anti-Fas-treated mouse. HPLC condition: solvent A, 0.01M NH₄OAc in water; solvent B, acetonitrile; gradient: 0-80% B in A over 40 min; flow rate 1.0 mL/min; C18 4.6×250 mm, 5-μm column. ^{99m}Tc radioactivity was detected with a NaI crystal radiodetector.

FIGS. 13 A & B depict analytical HPLC chromatograms of purified compounds S1 and S2. Absorbance was recorded at λ=254 nm.

FIGS. 14A & B depict analytical HPLC chromatograms of purified profluorophores 2 and 3. Absorbance was recorded at λ=254 nm.

FIGS. 15 A & B depict comparison of the stability of 2 and fluorescein diacetate in PBS and in cell culture medium containing 10% FBS. Time courses for the generation of fluorescence for both compounds were recorded continuously (λₑₓ=496 nm, λₑₘ=520 nm).

FIG. 16 shows the emission spectra of 2 incubated with caspase 3 for different time intervals. (λₑₓ=496 nm, λₑₘ=520 nm)

FIGS. 17 A, B & C provide L C-MS chromatograms (FIG. 17A) and MS spectra (FIGS. 17B &C) of 2 in the presence and absence of human caspase 3. The peaks at 17.85 min and 18.08 min correspond to the fragment R110-Asp-SAAC-Fmoc [(M+H)⁺=978.3941] and the parent compound 2 [(M+H)⁺=1478.5581], respectively. HPLC condition: solvent A, 0.1% TFA in water; solvent B, acetonitrile; gradient: 0-90% B in A over 30 min, flow rate 1.0 mL/min, C₁₈ 4.6×250 mm, 5-μm column.

FIGS. 18A & B show the uptake and efflux of ^{99m}Tc-chelate 4 and its fragment in DLD1 cells ^{99m}Tc-chelate accumulated significantly more in the cells than its fragment did. ^{99m}Tc-chelate 4 but not its fragment was washed out of the cells over time. Activity ratios of the cell pellet to medium are expressed as [cpm/g protein in pellet]/[cpm/g medium]. The experiments were performed in pentaplicate.

FIG. 19 shows the biodistribution of ^{99m}Tc-chelate 4 in anti-Fas- and PBS-treated mice. Biodistribution of ^{99m}Tc-chelate 4 at 2 h after intravenous injection in anti-Fas-treated and PBS-treated mice. *p=0.015. ID=injected dose. In particular, the executioner caspase 3 mediate the initiation and propagation of the apoptosis cascade. M. G. Gruner, Curr. Opin. Struct. Biol. 2000, 10, 649-655.

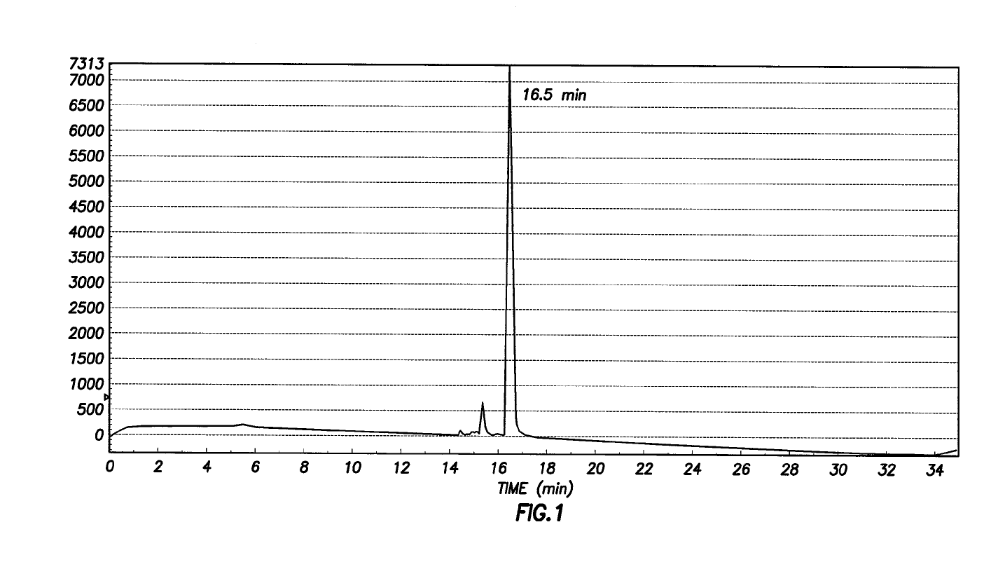

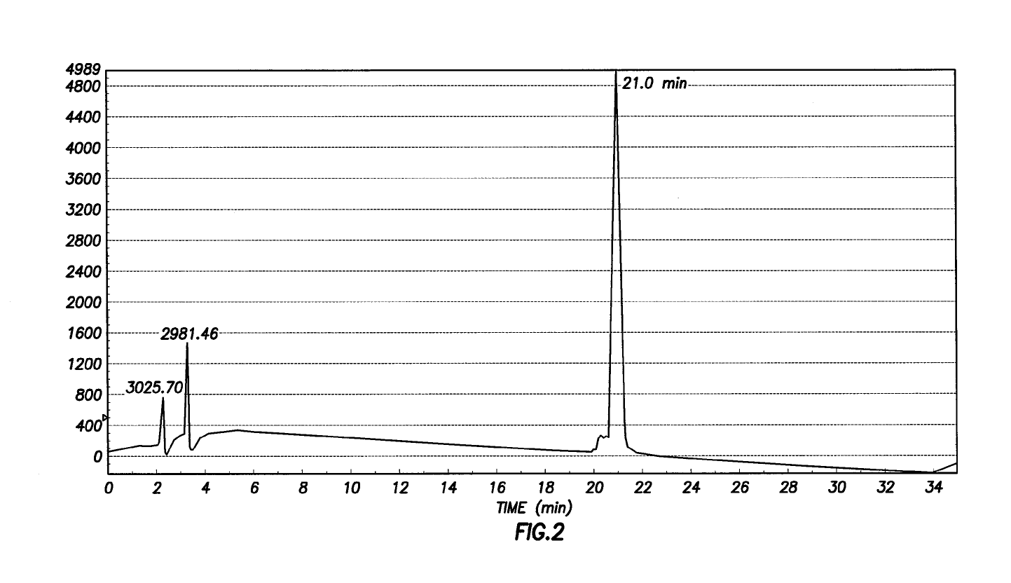

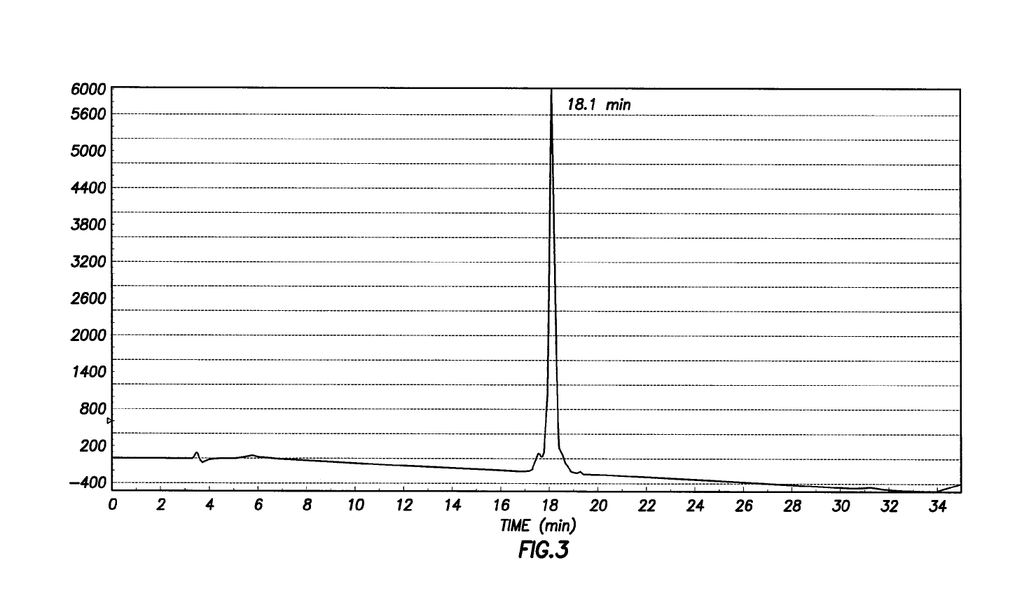

### DETAILED DESCRIPTION

Apoptosis, or programmed cell death, is a specialized form of cell death involved in a wide variety of physiological processes. S. J. Riedl, Y. Shi, Nat. Rev. Mol. Cell. Biol. 2004, 5, 897-907; Y. Shi, Nat. Struct. Biol. 2001, 8, 394-40. Many anticancer therapeutic regimens induce apoptosis. While noninvasive imaging techniques can markedly enhance the early evaluation and continuous monitoring of anticancer drug efficacy, to date, the ability to assess apoptosis using noninvasive imaging techniques has been very limited.

Caspases, a family of cysteinyl aspartate-specific proteases, play a central role in the regulation and execution of apoptosis. G. M. Cohen, Biochem. J. 1997, 326 (Pt 1), 1-16; N. A. Thornberry, Y. Lazebnik, Science 1998, 281, 1312-1316. One approach for real-time detection of apoptosis is to measure the activity of executioner caspases 3 and 7, while mediate initiation and propagation of the apoptosis cascade. M. G. Gruffer, Curr. Opin. Struct. Biol. 2000, 10, 649-655. Caspase 3 and 7 typically recognize the 4-amino acid peptide sequence Asp-Glu-Val-Asp (DEVD) and cleave their substrate at the C-terminal Asp residue. Fluorogenic DEVD conjugates that become fluorescent in the presence of caspase 3 and 7 have been used for the in vitro detection of apoptosis. Bullok, K; Piwnica-Worms, D. J. Med. Chem 2005, 48, 5404.

In particular, the executioner capase 3 mediates the initiation and propagation of the apoptosis cascade. M. G. Gruffer, Curr. Opin. Struct. Biol. 2000, 10, 649-655. Fluorogenic DEVD conjugates that become fluorescent in the presence of caspase 3 have been used for the in vitro detection of apoptosis using fluorescence microscopy. Two strategies have been explored for the noninvasive imaging of apoptosis. The first approach uses annexin V, a 36-KDa protein, as the imaging probe for apoptotic cells. J. L. Vanderheyden, G. Liu, J. He, B. Patel, J. F. Tait, D. J. Hnatowich, Nucl. Med. Biol. 2006, 33, 135-144; F. G. Blankenberg, P. D. Katsikis, J. F. Tait, R. E. Davis, L. Naumovski, K. Ohtsuki, S. Kopiwoda, M. J. Abrams, M. Darkes, R. C. Robbins, H. T. Maecker, H. W. Strauss, Proc. Natl. Acad. Sciences USA 1998, 95, 6349-6354; F. G. Blankenberg, P. D. Katsikis, J. F. Tait, R. E. Davis, L. Naumovski, K. Ohtsuki, S. Kopiwoda, M. J. Abrams, H. W. Strauss, J. Nucl. Med. 1999, 40, 184-191. Annexin V binds with high affinity to phosphatidylserine, which is externalized from the inner to the outer leaflet of the plasma membrane during the early stage of apoptosis. M. Mourdjeva, D. Kyurkchiev, A. Mandinova, I. Altankova, I. Kehayov, S. Kyurkchiev, Apoptosis 2005, 10, 209-217. The second approach is to measure the activity of specific intracellular enzymes involved in the apoptotic pathway.

Nuclear imaging offers high detection sensitivity, which makes it especially suited for tracking radiotracers used in the in vivo molecular imaging of apoptosis. C. M. M. Lahorte, J.-L. Vanderheyden, N. Steinmetz, C. Van De Wiele, R. A. Dierckx, G. Slegers, Eur. J. Nucl. Med. Mol. Imaging. 2004, 31, 887-919. However, neither positron emission tomography nor single-photon emission computed tomography can localize radiotracers at the cellular level because of low spatial resolution. An effective and economical imaging strategy is to develop multimodality imaging probes that allow the extraction of as much diagnostic information as possible from each examination.

Technetium-99m (^{99m}Tc) is extensively used radiometal in nuclear medicine radiodiagnostics. L. Biancone, O, Schillaci, F. Capoccetti, R. M. Bozzi, D. Fina, C. Petruzziello, A. Geremia, G. Simonetti, F. Pallone, Am. J. Gastroenterol. 2005, 100, 344-354; J. De Boer, R. H. J. A. Slart, P. K. Blanksma, A. T. M. Willemsen, P. L. Jager, A. M. J. Paans, W. Vaalburg, D. A. Piers, Nucl. Med. Commun. 2003, 24, 251-257; W. C. Lavely, S. Goetze, K. P. Friedman, J. P. Leal, Z. Zhang, E. Garret-Mayer, A. P. Dackiw, R. P. Tufano, M. A. Zeiger, H. A. Ziessman, J. Nucl. Med. 2007, 48, 1084-1089. Rhenium (a mixture of ¹⁸⁵Re and ¹⁸⁷Re) generally produces complexes with physical and biodistribution properties similar to those formed with ^{99m}Tc and is often used as a nonradioactive alternative to ^{99m}Tc for structural characterization. S. R. Bayl), C. L. Fisher, T. Storr, M. J. Adam, C. Orvig, Bioconjug. Chem. 2004, 15, 923-926. Both ^{99m}Tc and Re form stable complexes with a lipophilic amino acid derivative, Lys[di(2-pyridinemethyl)]-CO₂H, also termed single-amino acid chelate (SAAC). M. K. Levadala, S. R. Banerjee, K. P. Maresca, J. W. Babich, J. Zubieta, Synthesis 2004, 1759-1766 K. A. Stephenson, S. R. Banerjee, O. O. Sogbein, M. K. Levadala, N. McFarlane, D. R. Boreham, K. P. Maresca, J. W. Babich, J. Zubieta, J. F. Valliant, Bioconjug. Chem. 2005, 16, 1189-1195. Rhodamine-based fluorogenic substrates have a low background signal in their uncleaved state and the ability to unveil the majority of the fluorescence after cleavage of one of the two amide bonds.

Membrane-permeable probes having a caspase 3 substrate, a fluorogenic dye and a radionuclide are provided herein. Cleavable rhodamine-based DEVD substrate linked to Re-SAAC or ^{99m}Tc-SAAC chelate is suitable candidates for detecting caspase activity both in vitro using fluorescence microscopy and in vivo using nuclear imaging. The lipophilic nature of rhodamine 110 (R110) and Re/^{99m}Tc-SAAC facilitates the cellular uptake of the DEVD substrate. Once the amide bond between DEVD and rhodamine is cleaved, the amino group on the R110-Re/^{99m}Tc-SAAC fragment in the cytosol is expected to be protonated and to have increased hydrophilicity and increased cellular retention.

As shown in Scheme 1 immediately below, SAAC is linked to R110 through one of its two aromatic amines, and the other amine in R110 was linked to DEVD peptide at its C terminus. Synthesis of Ac-DEVD-R110-D-SAAC-Fmoc (2) was initiated by coupling R110 with 10 equiv in excess of Fmoc-Asp(OBu-t)-CO₂H followed by removal of the Fmoc-protecting group. The resulting product [Asp (OBu-t)]₂-R110 was obtained with 90% yield. Sequential condensation with 1.5 equiv of Fmoc-protected SAAC (Fmoc-SAAC-CO₂H) and 2 equiv of tripeptide Ac-Asp(OBu-t)-Glu(OBu-t)-Val-OH followed by removal of t-butyl protecting groups with trifluoroacetic acid gave Ac-Asp-Glu-Val-Asp-R100-SAAC (also referred to as DEVD-R100-SAAC) marked as 2 on Scheme 1 below with an overall yield of 8%. The profluorophore 2 was stable in both PBS and culture medium supplemented with serum. (See Examples herein). This is important for 2 and its corresponding ^{99m}Tc-chelate to be useful as imaging agents because premature hydrolysis of the imaging probes can raise background signal levels and deteriorate imaging quality.

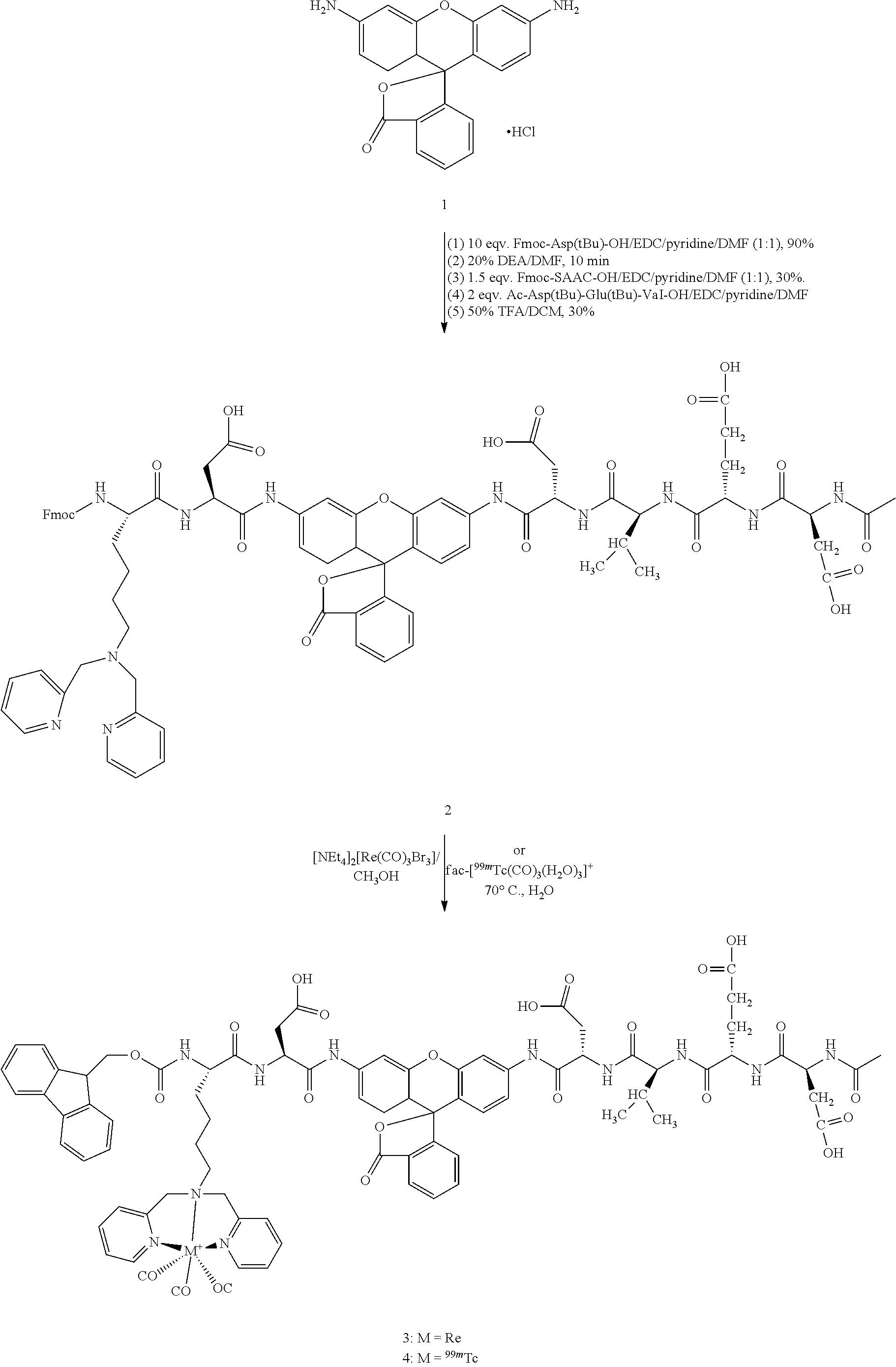

Rhenium was chelated to 2 using a stoichiometric amount of the Re (I) tricarbonyl precursor (NEt₄)₂[Re(CO)₃Br₃] in methanol to give 3 in quantitative yield. T. Storr, Y. Sugai, C. A. Barta, Y. Mikata, M. J. Adam, S. Yano, C. Orvig, Inorg. Chem. 2005, 44, 2698-2705. The structure of 3 was verified by liquid chromatography-mass spectroscopy (LC-MS).

Compound 2 was completely degraded when it was incubated with caspase 3, as verified by increase in fluorescence signal upon exposure to caspase 3, and by LC-MS analysis showing that the hydrolytic loss of R110-Asp-SAAC-Fmoc fragment occurred selectively at the C-terminus of DEVD peptide. To ascertain that 2 and its Re-chelate, 3, could be cleaved by apoptotic cells and activate the fluorescent signal, both compounds were incubated with either viable human colon cancer DLD1 cells or apoptotic DLD1 cells that had been treated with tumor necrosis factor-related apoptosis-inducing ligand (TRAIL) for 2 h at 37° C. Fluorescence microscopic images of cells incubated with 3 are shown in FIG. 8. TRAIL induced significant apoptosis, as revealed by extensive staining of TRAIL-treated DLD1 cells with annexin V-Alexa Fluor 594 (FIG. 8B, 8C). Cleavage of R110-D-SAAC-Re(CO)₃ and subsequent activation of fluorescent signal from 3 was visualized in apoptotic cells (FIG. 8A) but not in untreated viable cells (FIG. 8B). Treatment with both TRAIL and a selective caspase 3 inhibitor, Ac-DEVD-CHO, significantly reduced the fluorescent signal from 3, although Ac-DEVD-CHO did not inhibit the apoptosis of DLD1 cells, as indicated by their strong positive annexin V staining (FIG. 8C). These results indicated that 3 was specifically cleaved by caspases 3 in apoptotic cells. A Z-stack image of TRAIL-treated DLD1 cells at higher magnification showed that the fluorescent signal was activated in the cytoplasm (FIG. 8D), providing direct evidence that 3 was cell permeable and that the degradation product was retained in the cells. Similar findings were observed for the nonchelated profluorophore 2.

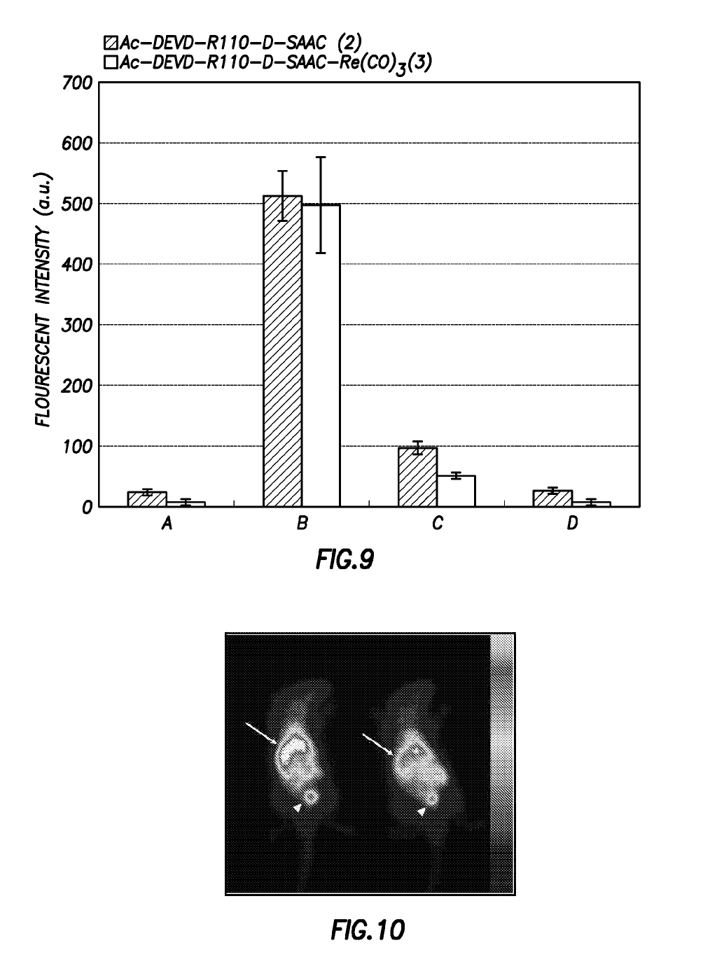

To test whether 2 and 3 could diffuse out of cells, each compound was exposed to DLD1 cells for 2 h. The cells were then washed and incubated in drug-free culture medium for 2 or 24 h. If the test compound diffused out of the cells during the incubation period, the fluorescent signal resulting from subsequent treatment with TRAIL would decrease with increasing incubation time in drug-free medium owing to the reduced concentration of each compound entrapped in the cells. This is exactly what we observed (FIG. 9). Our data indicated that uptake of 2 and 3 in viable tumor cells was a reversible process, a condition necessary for reduced background signal and satisfactory nuclear imaging applications.

We next labelled 2 with ^{99m}Tc to afford ^{99m}Tc-chelate 4, which shares chemical and biological properties similar to those of Re-chelate 3. Compound 2 was treated with [^{99m}Tc—(CO)₃H₂O]⁺, which was generated from ^{99m}TcO₄⁻ using commercially available carbonyl labelling kits (Mallinckrodt, St. Louis, Mo.). The labelling efficiency was >95%. HPLC analysis revealed that ^{99m}Tc-chelate 4 had a retention time (t_{R}=31.2 min) identical to that of Re-chelate 3 (t_{R}=30.9 min), indicating that 4 had the same structure as 3. The stability of ^{99m}Tc-chelate 4, Ac-DEVD-R110-D-SAAC-^{99m}Tc(CO)₃, was evaluated using a ligand challenge method, in which a large excess of cysteine and histidine was used to compete for the complex's tendency to undergo transchelation. Chelate 4 was stable as no transchelation was observed after samples were incubated at 37° C. for up to 5 h.

We examined whether the ^{99m}Tc-chelate of 2 (4) could be retained by TRAIL-treated apoptotic DLD1 cells. However, because a fraction of treated cells were detached from the microwell plate and some late-stage apoptotic cells lost membrane integrity during the time course of the induction, large variations in experimental data were observed. The results of radiotracer uptake study in apoptotic cells were not conclusive. To provide evidence to differential cellular uptake and wash-out between ^{99m}Tc-chelate 4 and its fragment R110-D-SAAC-^{99m}Tc(CO)₃, each radiotracer was incubated with DLD1 cells, and their retention in the cells was measured. ^{99m}Tc-chelate 4 had significantly higher uptake in DLD1 cells than did R110-D-SAAC-^{99m}Tc(CO)₃ after 1-2 h of incubation. On the other hand, while chelate 4 was gradually washed out, its fragment was trapped in the cells. These data indicate that the cleaved fragment R110-D-SAAC-^{99m}Tc(CO)₃ is less permeable to plasma membrane as compared to its parent substrate ^{99m}Tc-chelate 4.

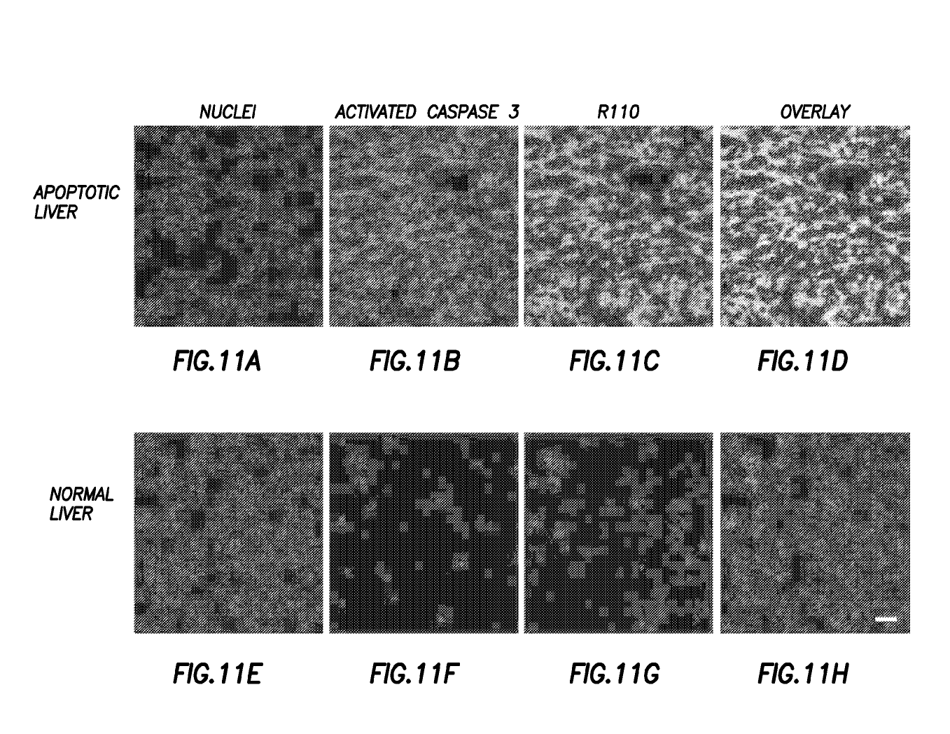

We used a murine liver apoptosis model to evaluate whether chelate 4 could be used to image apoptosis in vivo. F. G. Blankenberg, J. F. Tait, R. E. Davis, L. Naumovski, K. Ohtsuki, S. Kopiwoda, M. J. Abrams, M. Darkes, R. C. Robbins, H. T. Maecker, H. W. Strauss, Proc. Natl. Acad. Sciences USA 1998, 95, 6349-6354; F. G. Blankenberg, P. D. Katsikis, J. F. Tait, R. E. Davis, L. Naumovski, K. Ohtsuki, S. Kopiwoda, M. J. Abrams, H. W. Strauss, J. Nucl. Med. 1999, 40, 184-191. Male Balb/c mice (10-12 weeks old, weight 20-25 g) were injected intravenously with an anti-Fas monoclonal antibody (10 μg/mouse) to induce apoptosis in the liver. Two hours later, ^{99m}Tc-chelate 4 [300 μCi (11.1 MBq), 20 peptide in 0.2 ml] was injected intravenously. Radionuclide imaging was acquired 120 min after the injection of 4. Significantly higher uptake of 4 was seen in anti-Fas-treated apoptotic liver than in the PBS-treated control mice. ^{99m}Tc-chelate 4 was cleared through the renal route as indicated by the distribution of radioactive species into the bladder (FIG. 10).

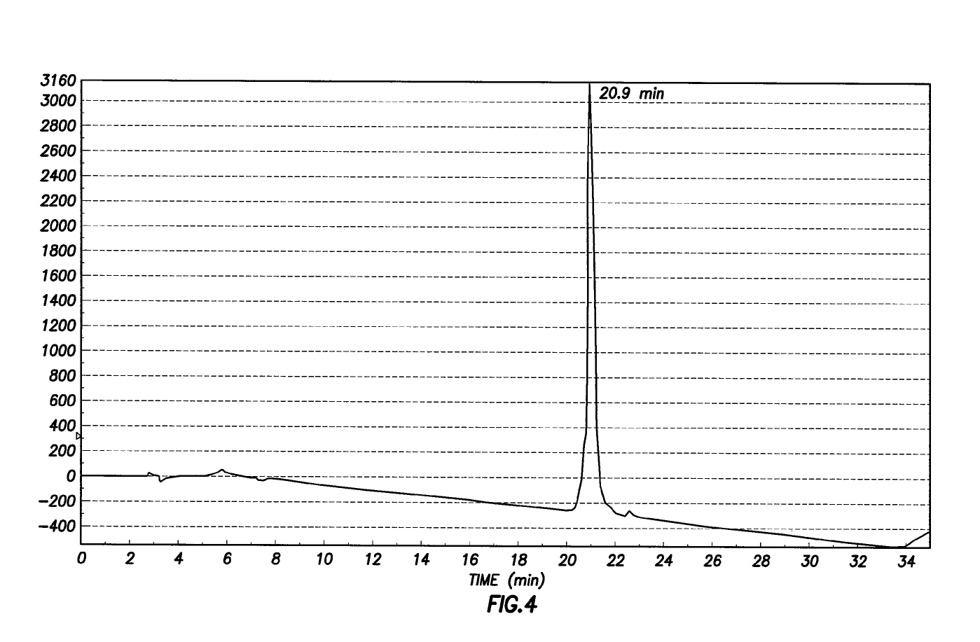

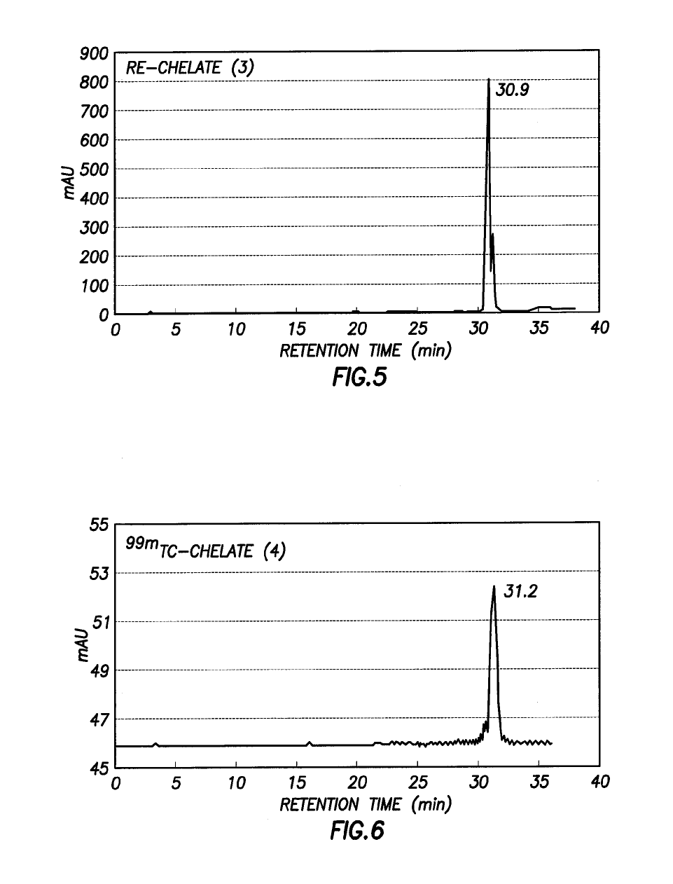

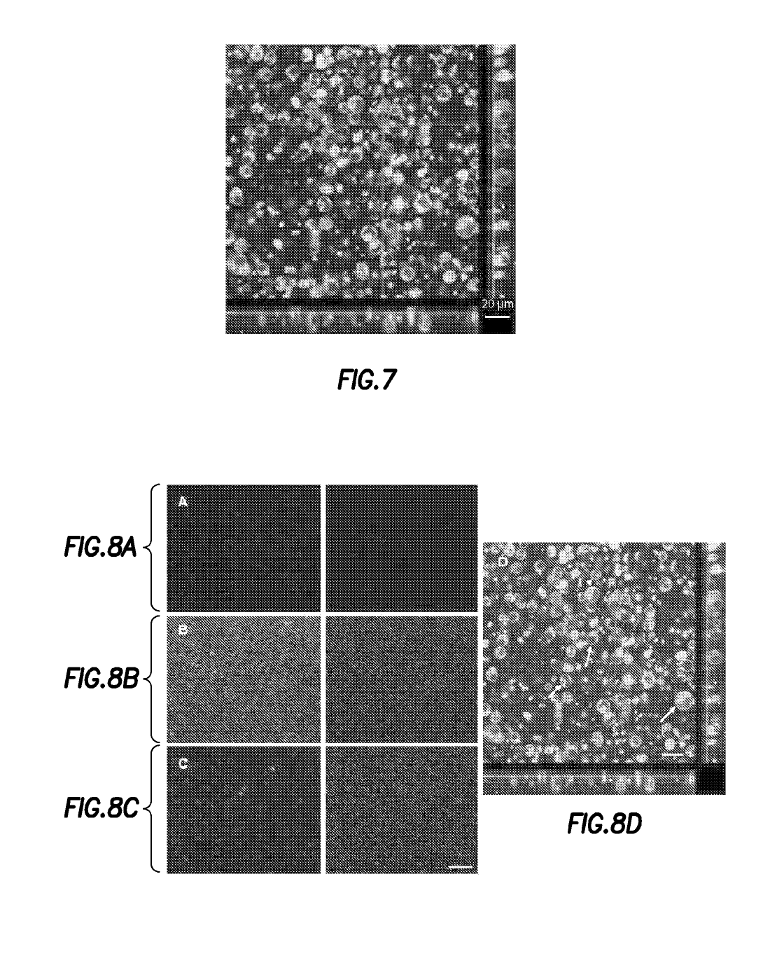

To confirm that 4 was activated by caspases 3 and retained in apoptotic liver, the liver tissues were dissected and cryosectioned to 5-μm slides after gamma imaging. Activated caspase 3 was immunohistochemically stained with a rabbit anti-mouse monoclonal antibody against activated caspase 3, and cell nuclei were counterstained with Hoechst 33342. As shown in FIG. 4, anti-Fas-treated liver but not PBS-treated liver displayed extensive staining for activated caspase 3. Importantly, cleavage of the fluorogenic ^{99m}Tc-chelate 4 was shown only in the apoptotic liver but not in the normal liver, indicating that 4 was cleaved by caspase 3 in vivo (FIG. 11).

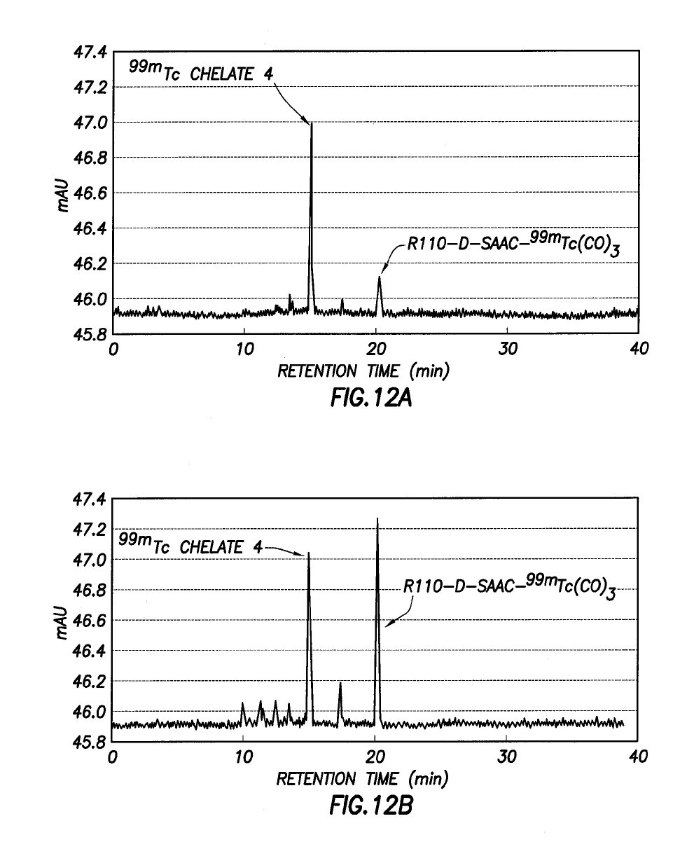

Cleavage of R110 dye from the DEVD peptide in 4 does not necessarily imply that the radionuclei component SAAC-^{99m}Tc(CO)₃ is associated with the dye. To confirm that SAAC-^{99m}Tc(CO)₃ remains linked to R110 in the apoptotic liver after cleavage of 4 by caspase 3, we analyzed the chromatograms of liver extracts from mice 2 h after intravenous injection of 4. Whereas only trace amount of the fragment R110-D-SAAC-^{99m}Tc(CO)₃ was detected from the liver of PBS-treated mouse, substantially more R110-D-SAAC-^{99m}Tc(CO)₃ was detected in the liver of anti-Fas-treated mouse (FIG. 12). The ratios of area integrals between peaks representing R110-D-SAAC-^{99m}Tc(CO)₃ and 4 were 0.20 and 1.48 in PBS-treated liver and anti-Fas-treated liver, respectively. These data indicate that ^{99m}Tc-chelate 4 was cleaved selectively at the amide bond between Ac-DEVD and R110-D-SAAC-^{99m}Tc(CO)₃, and that the corresponding degradation product R110-D-SAAC-^{99m}Tc(CO)₃ was retained in the apoptotic liver in its intact form. Because fluorescent signal from cleaved products of 4 co-localized with activated caspase 3 (FIG. 11), we conclude that the increased radioactivity in the liver of anti-Fas-treated mice observed in the gamma imaging study is attributed to the activation of caspase 3.

### Example 1

Synthesis of Ac-DEVD-R110-D-SAAC-Fmoc (2) To a solution of Ac-Asp(OBu-t)-Glu(OBu-t)-Val-CO₂H (288 mg, 0.56 mmol) in anhydrous 1:1 mixture of DMF and pyridine (5 mL) was added N-(3-dimethylaminopropyl)-N-ethylcarbodiimide (110 mg, 0.56 mmol). The solution was stirred at room temperature for 30 min, and then (Asp(OBu-t)-R110-Asp(OBu-t)-SAAC-Fmoc (S2 of Example 2 Below) was added (116 mg, 0.113 mmol). The solution was stirred at room temperature for 3 days. After removal of the solvent under vacuum, the residue was dissolved in ethyl acetate and washed with saturated NaHCO₃ (3 times) and brine (3 times), and the organic solution was dried over Na₂SO₄. Ethyl acetate was removed under vacuum, and the solid was treated with 50% TFA in dichloromethane for 30 min to remove the t-butyl-protecting groups. The product was purified by preparative HPLC to yield 50 mg (30%) of 2. ¹H NMR (MeOD₃): [symbol] 1.00 (m, 2H), 1.45 (br, 2H), 1.65-2.50 (m, 10H), 3.25-3.35 (m, 20H), 4.09-4.62 (m, 10H), 6.60-6.82 (m, 2H), 7.10-8.10 (m, 22H), 8.62-8.64 (m 2H). High resolution MS calcd. for C₇₇H₈₀N₁₁O₂₀ (M+H) 1478.5503, found 1478.5657. HPLC: t_{R}=18.1 min (solvent A, 0.1% TFA in water; solvent B, acetonitrile; gradient: 0-90% B in A over 30 min, flow rate 1.0 mL/min, C18 4.6×250 mm, 5-μm column).

Synthesis of Re-chelate (3) The Re chelate was prepared in quantitative yield by adding a stoichiometric amount of (NEt₄)₂[Re(CO)₃Br₃] in methanol to 2. High resolution MS calcd. for C₈₀H₇₉N₁₁O₂₃Re (m/z) 1748.4908, found 1748.5588. HPLC: t_{R}=20.9 min (solvent A, 0.1% TFA in water; solvent B, acetonitrile; gradient: 0-90% B in A over 30 min, flow rate 1.0 mL/min, C18 4.6×250 mm, 5-μm column). t_{R}=30.9 min (solvent A, 0.1% TFA in water; solvent B, acetonitrile; gradient: 10-50-80% B in A over 36 min, flow rate 1.0 mL/min, C18 4.6×250 mm, 5-μm column). The same chromatographic conditions were used in the analysis of ^{99m}Tc-chelate.

Synthesis of ^{99m}Tc-chelate (4) [^{99m}Tc(CO)₃(H₂O)₃]⁺¹ was prepared using the following general procedure: 1.0 mL of ^{99m}TcO₄⁻ (5 mCi) was added to commercially available Isolink carbonyl kits (Mallinckrodt, St. Louis, Mo.). The solution was heated in a water bath at 100° C. for 20 min. The solution was then cooled for 5 min and vented. Aliquots of 1N HCl (˜120 μL) were added to the above solution to adjust the pH to 6-7 and to decompose any residual boranocarbonate. Into a sealed vial containing 10-20 μg of 2 in distilled water was added 0.5 mL of ^{99m}Tc—(CO)₃(H₂O)₃ (2 mCi). The vial was heated for 30 min at 75° C. After the sample was cooled, the chelate were analyzed using HPLC. The labeling efficiency was greater than 95%. HPLC: t_{R}=31.2 min. The following conditions were used for HPLC analysis: solvent A, 0.1% TFA in water; solvent B, acetonitrile; gradient: 10-50-80% B in A over 36 min; flow rate 1.0 mL/min; C18 4.6×250 mm, 5-μm column; UV detector: Agilent 1100 210 set at 254 nm; radioactivity detector: Bioscan flow-count radio-HPLC detector.

Fluorescence Microscopy Human epithelial cells from colorectal adenocarcinoma (DLD1) were seeded (1×10⁴/well) in a 96-well plate 1 day before experiments. Apoptosis was induced by treating cells with TRAIL (100 μL, 250 ng/mL) for 2 h at 37° C. Both apoptotic and viable cells were incubated with 100 μL of 2 or 3 in (final concentration, 50 μM) for 2 h at 37° C. The cells were washed and stained with Annexin V-Alexa Fluor 594. After washing with PBS, the cells were transferred into Lab-Tek II chambered cover glass and visualized under Olympus Fluoview FV1000 confocal laser scanning microscope (FV1-ASW, Olympus, Melville, N.Y.) equipped with fluorescein isothiocyanate (wavelength: 495/519 nm) and Texas red filters (wavelength: 589/615 nm). In separate wells, TRAIL was added in the presence of 50 μM of Ac-DEVD-CHO to inhibit caspase 3 activity.

Gamma Imaging and Biodistribution For γ-imaging study, two male Balb/c mice (10-12 weeks old, weight 20-25 g) (Charles River, Wilmington, Mass.) were injected intravenously with a purified anti-Fas monoclonal antibody (10 μg/mouse) (Pharmingen, San Diego, Calif.). Two mice injected with PBS were used as a control group. Two hours later, ^{99m}Tc-chelate 4 [300 μCi (11.1 MBq), 20 μg peptide in 0.2 mL] was injected intravenously. Radionuclide imaging was acquired 120 min after the administration of ^{99m}Tc-chelate 4 with the following parameters: matrix, 512×512 pixels; energy peak, 140 keV (15%). For biodistribution study, Balb/c mice were divided into 2 groups consisting of 7 mice each. Mice in the first group were subjected to anti-Fas antibody treatment (10 μg/mouse); mice in the second group were injected with PBS solution (200 μL/mouse). Two hours after treatments, the mice were injected with ^{99m}Tc-chelate 4 intravenously through the tail vein at a dose of 2 nmol/mouse (50 μCi/mouse). Mice were killed with CO₂ exposure 2 h after radiotracer injection. The organs of interest were excised and weighed, and radioactivity was counted in a gamma counter. The stomach and intestines were emptied of food contents prior to radioactivity measurements. The percentage of injected dose per gram (% ID/g) was calculated by dividing the % ID/organ by the weight of the tissue.

### Example II

Amino acid derivatives were purchased from Novabiochem (San Diego, Calif.), Bachem (Torrance, Calif.), and Chem-Impex International (Wood Dale, Ill.). R110 was obtained from Acros (Morris Plains, N.J.). Other chemicals were obtained from Aldrich (St. Louis, Mo.) and were used as received. Reagent-grade solvents were used without further purification unless otherwise specified. Recombinant human TRAIL was purchased from Millipore (Billerica, Mass.). Alexa Fluor 594-annexin V conjugate, fetal bovine serum, and RPMI 1640 culture medium were purchased from Invitrogen (Carlsbad, Calif.). Caspase 3 and its inhibitor Ac-DEVD-CHO were purchased from Sigma (St. Louis, Mo.). Liquid chromatography-mass spectroscopy was performed on an Agilent LC-MSD-TOF system (Santa Clara, Calif.) in the positive ion mode using the electrospray ionization method. 1H and 13C NMR spectra were recorded on a Bruker DRX-500 spectrometer (Billerica, Mass.). Preparative high performance liquid chromatography (HPLC) was run on an Agilent 1200 system (C-18, Vydac, 10×250 mm, 10 μm) with water and acetonitrile as the mobile phase at a flow rate of 10 mL/min.

Fmoc-protected single-amino-acid chelate (Fmoc-SAAC-CO₂H), Fmoc-Lys[di(2-pyridinemethyl)]-CO₂H, was synthesized according to the method of Levadala et al. M. K. Levadala, S. R. Banerjee, K. P. Maresca, J. W. Babich, J. Zubieta, Synthesis 2004, 1759. Tripeptide Ac-Asp(OBu-t)-Glu(OBu-t)-Val-COOH was synthesized by Fmoc solid-phase peptide chemistry using 2-chlorotrityl resin as the solid support. The peptide was cleaved from the resin with dilute trifluoroacetic acid (TFA). The organometallic precursor (NEt₄)₂[Re(CO)₃Br₃] was synthesized according to previously published procedures. R. Alberto, A. Egli, U. Abram, K. Hegetschweiler, V. Gramlich, P. A. Schubiger, J. Chem. Soc., Dalton Trans. 1994, 2815.

Synthesis of [Asp(OBu-t)]₂-R110 (S1) To a solution of Fmoc-Asp(OBu-t)-CO₂H (1.65 g, 4 mmol) and 1-[3-(dimethylamino)propyl]-3-ethylcarbodiimide hydrochloride (EDC) (0.788 g, 4 mmol) in 5 mL of dimethylformamide (DMF)/pyridine (9:1, v/v) was added R110 (200 mg, 0.55 mmol). The reaction mixture was stirred at room temperature under nitrogen protection overnight. The solvent was evaporated under vacuum, and the residue was redissolved in ethyl acetate. The organic solvent was washed sequentially with saturated aqueous NaHCO₃ solution (3 times) and brine (3 times) and was dried over anhydrous Na₂SO₄. After removal of ethyl acetate under vacuum, the residue was treated with 20% diethylamine in DMF for 20 min to remove the Fmoc protecting group. The product was purified using silica column chromatography eluted with CHCl₃/MeOH (10:1, v/v) to yield 350 mg (90%) of S1 as an orange oil. ¹H NMR (MeOD₃): δ 1.48 (s, 18H). 2.90-3.11 (m, 4H), 4.38 (dd, J₁=8.1, Hz, J₂=4.2 Hz, 2H), 6.77 (d, J=8.4 Hz, 2H), 7.22 (m, 3H), 7.44 (m, 1H), 7.70-7.90 (m, 4H), 8.05 (m 1H); HRMS calcd for C₃₆H₄₁N₄O₉ (M+H) 673.2874, found 673.2892. HPLC: t_{R}=16.5 min (solvent A, 0.1% TFA in water; solvent B, acetonitrile; gradient: 0-90% B in A over 30 min, flow rate 1.0 mL/min, C18 4.6×250 mm, 5-μm column).

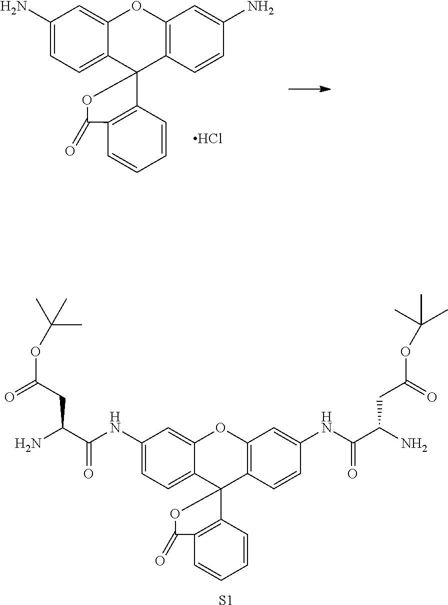

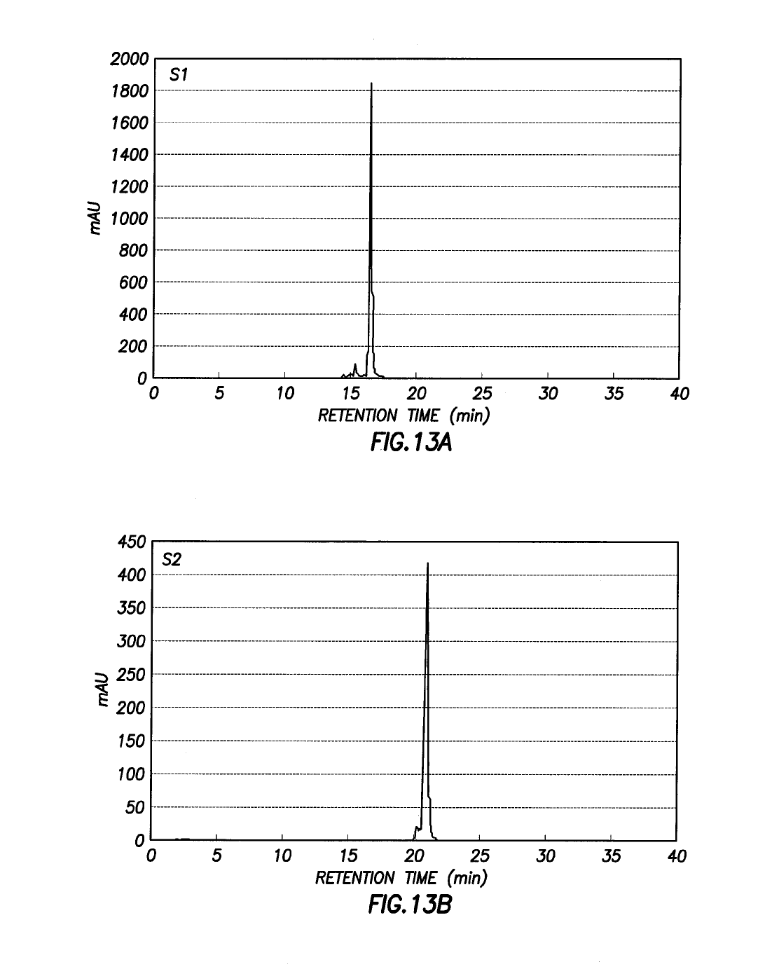

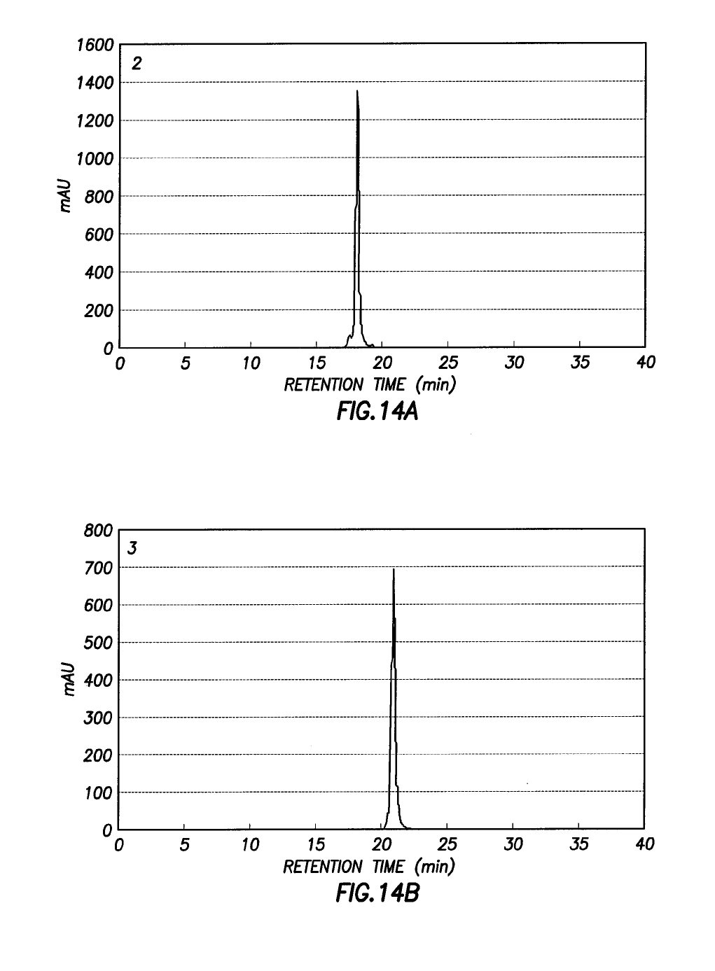

Synthesis of Asp(OBu-t)-R110-Asp(OBu-t)-SAAC-Fmoc (S2) To a solution of Fmoc-SAAC-CO₂H (473 mg, 0.86 mmol) and EDC (170 mg, 0.86 mmol) in 2 mL of a mixture of DMF and pyridine (9:1, v/v) was added S1 (385 mg, 0.57 mmol). The reaction mixture was stirred at room temperature, and the progress of the reaction was monitored by liquid chromatography-mass spectroscopy (LC-MS). Once the peak corresponding to monosubstituted product became maximum, the reaction was stopped by removing the solvent, and the residue was purified by preparative HPLC to yield 200 mg (30%) of S2. ¹H NMR (MeOD₃): δ 1.39 (s, 9H), 1.49 (s, 9H), 1.71 (br, 4H), 2.77-3.14 (m, 6H), 3.31-3.36 (m, 6H), 4.09-4.56 (m, 8H), 6.66-6.80 (m, 2H), 7.15-8.07 (m, 22H), 8.66-8.68 (m 2H). High resolution mass spectra-electron spray ionization (HRMS-ESI): calcd for C₆₉H₇₃N₈O₁₂ (M+H) 1205.5348, found (M+H) 1205.5498. HPLC: t_{R}=21.0 min (solvent A, 0.1% TFA in water; solvent 13, acetonitrile; gradient: 0-90% B in A over 30 min, flow rate 1.0 mL/min, C18 4.6×250 mm, 5-μm column). Chromatograms of S1 and S2 are shown in FIGS. 13 A & B.

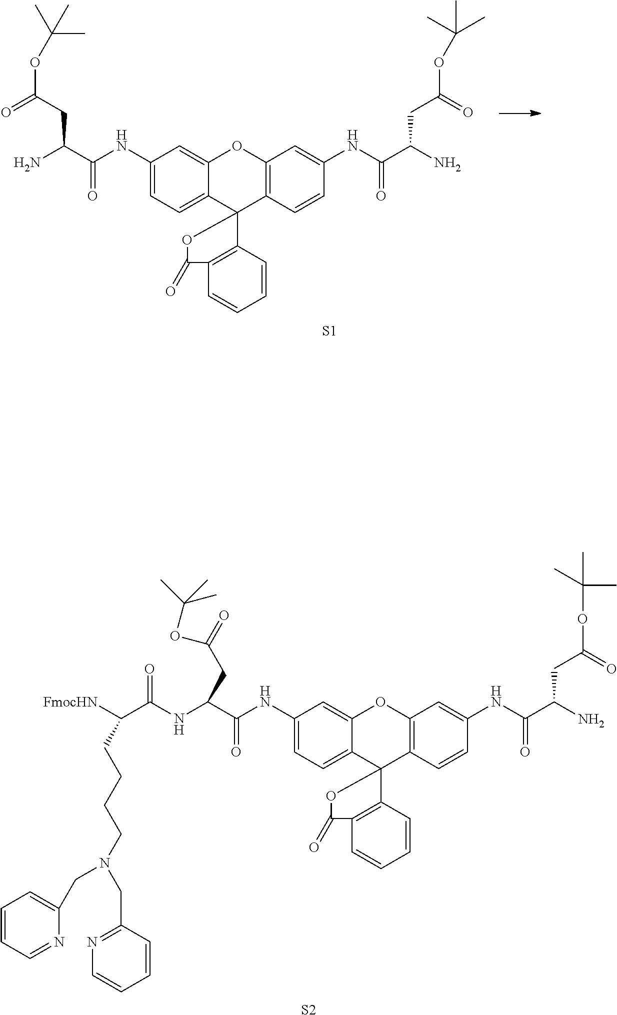

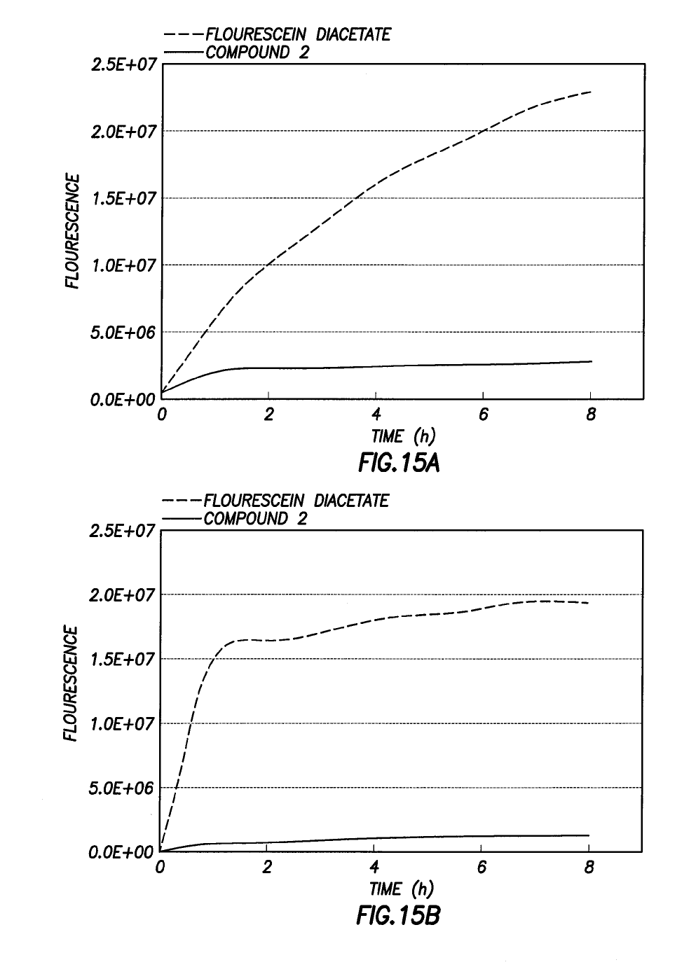

Stability of DEVD profluorophore 2 in PBS and serum-containing culture medium For the stability assay, the generation of fluorescence was recorded continuously at room temperature for 8 h for a solution of 2 (10 μM) or fluorescein diacetate (10 μM) in 3 mL of PBS using a Fluorolog-3 fluorometer (Jobin Yvon, Edison, N.J.) at λₑₓ=496 nm. The procedure was repeated for a solution of 2 (1 μM) or fluorescein diacetate (1 μM) in Dulbecco's modified Eagle medium supplemented with 10% (v/v) fetal bovine serum. Cleavage of amide bond linking R110 to DEVD or hydrolysis of fluorescein diacetate would result in increased fluorescence signal. FIGS. 15 A & B shows that the profluorophore 2 has remarkable stability in both PBS and culture medium supplemented with serum. In contrast, fluorescein diacetate suffered relatively rapid hydrolysis in both solutions.

Stability of ^{99m}Tc-chelate 4 The stability of ^{99m}Tc-chelate 4, Ac-DEVD-R110-D-SAAC-Fmoc-^{99m}Tc(CO)₃, was evaluated using a ligand challenge method. A large excess of competing donors cysteine and histidine was used as a means of evaluating the complex's tendency to undergo transchelation. Aliquots of 30 μL of ^{99m}Tc-chelate 4 were added to 270 μL of 0.01 M cysteine or 0.01 M histidine solution in PBS. The samples were incubated at 37° C. and analyzed by HPLC at 1- and 5-h intervals. No transchelation was observed for ^{99m}Tc-chelate 4 at both time points.

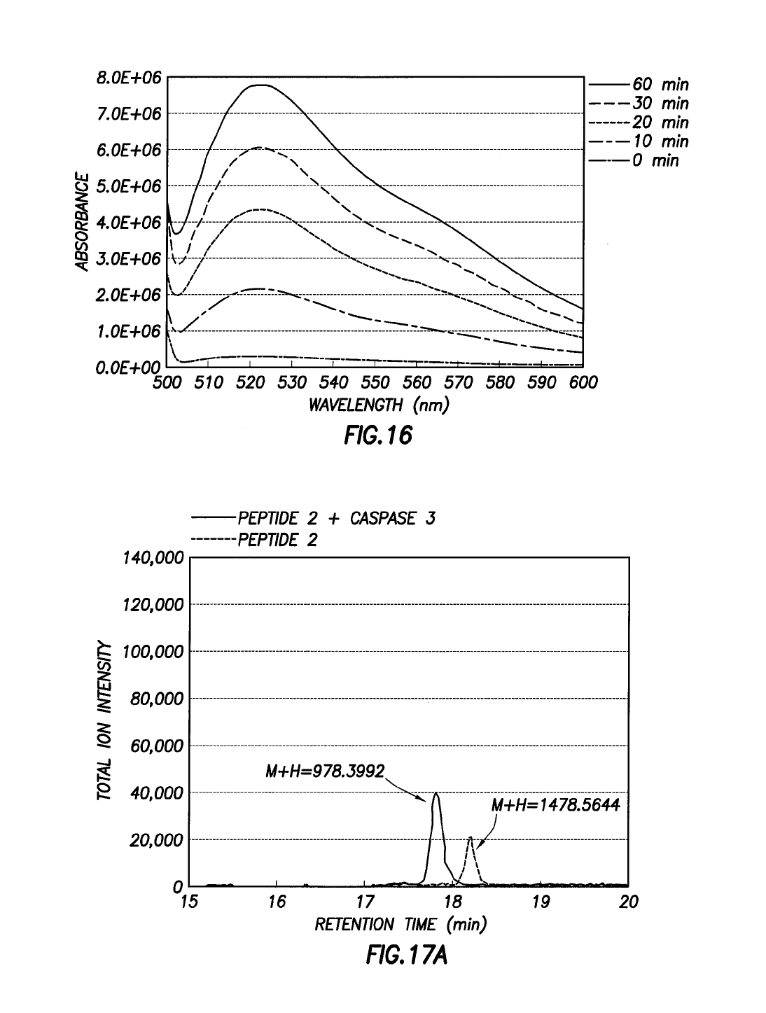

Cleavage by caspase 3 Human caspase 3 (Sigma-Aldrich) (5 μg) was reconstituted with 50 μL of deionized water (100 μg/mL) and stored in aliquots at −70° C. Before its use, an aliquot of reconstituted caspase 3 was thawed and diluted 200-fold in 1× assay buffer to a final concentration of 0.5 μg/mL. Five microliters of diluted caspase 3 was then added into a solution of 2 (22 μM) in 3 mL 1× assay buffer (20 mM HEPES [pH 7.4], with 2 mM ethylenediaminetetraacetic acid, 0.1% 3-[(3-cholamidopropyl)dimethylammonio]-1-propanesulfonate, and 5 mM dithiothreitol). The reaction mixture was incubated in cuvet in dark at room temperature and the emission spectra were recorded at intervals of 0, 10, 20, 30 and 60 min on a Fluorolog 3 fluorometer (HORIBA, Edison, N.J.) with an excitation wavelength (λₑₓ) of 496 nm. The fluorescence spectra of 2 exhibited near-baseline emission without exposure to caspase 3. Introduction of caspase 3 to the solution resulted in a large increase in fluorescence over a period of 60 min (FIG. 16), indicating cleavage of the amide bond between DEVD peptide and R110 in 2 by the enzyme.

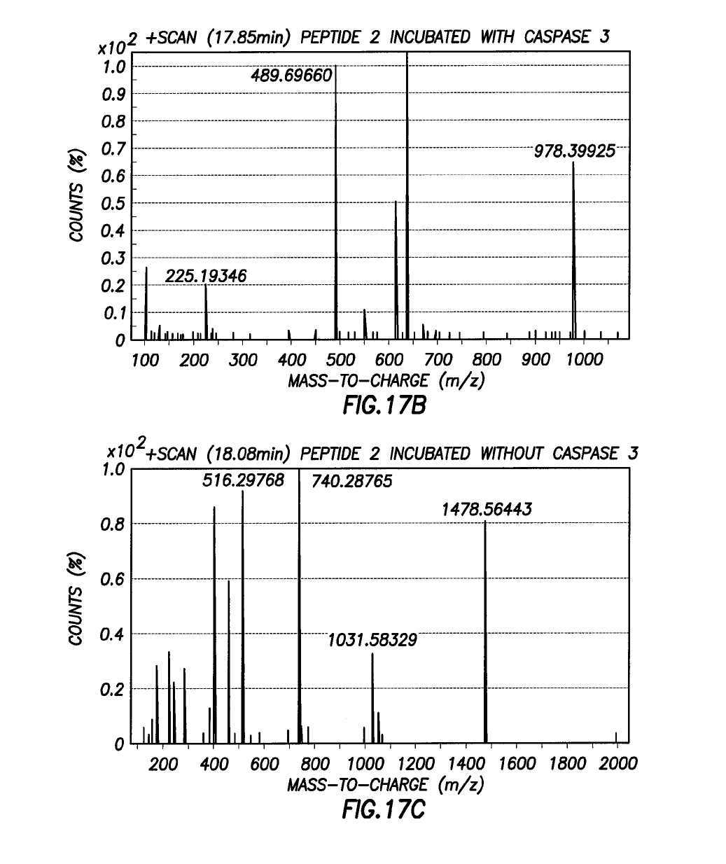

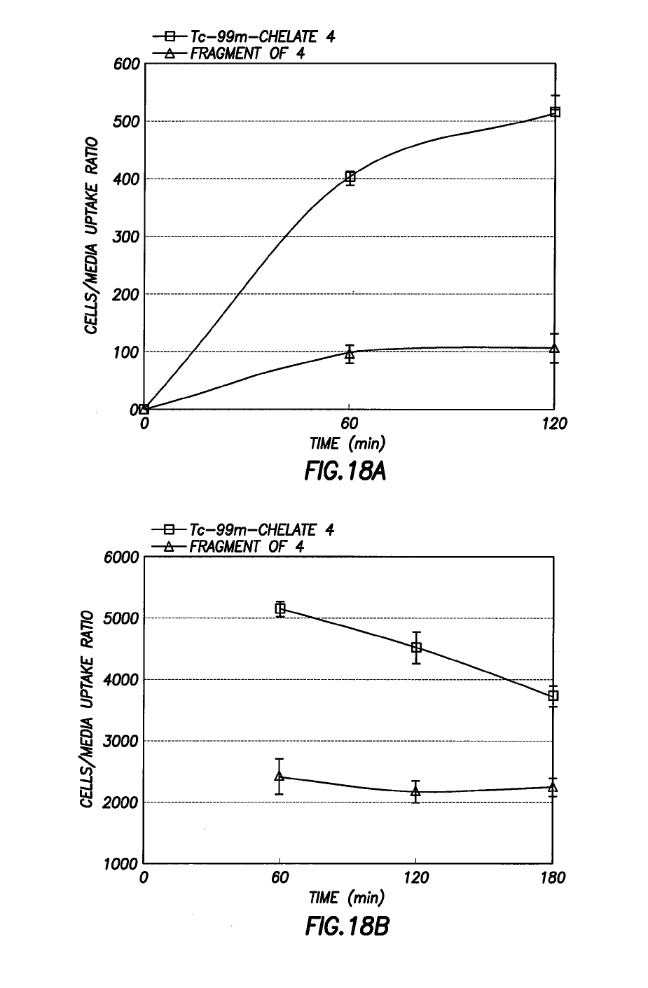

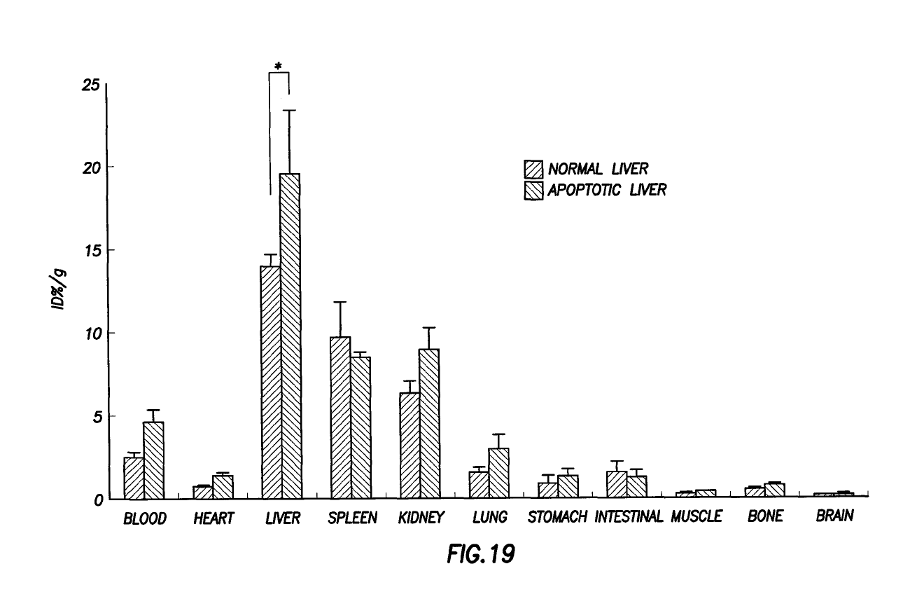

To further confirm cleavage of 2 by caspase 3, 5 μL of caspase 3 (0.5 μg/mL) was mixed with 10 μL of 1 mM solution of 2 dissolved in 1× assay buffer. After 1 h at room temperature, the reaction mixture was injected into LC-MS system. The mass difference of the parent and the fragment ions indicates that the hydrolytic loss of the C-terminal R110-Asp-SAAC-Fmoc occurred selectively at the amide bond between DEVD and R110 (FIGS. 17 A, B, & C).

Fluorescence microscopy Human epithelial cells from colorectal adenocarcinoma (DLD1) were obtained from American Type Cell Culture (Manassas, Va.). Cells were seeded (1×10⁴/well) in a 96-well plate (Corning, Lowell, Mass.) supplemented with RPMI-1640 medium plus 10% fetal bovine serum (FBS) 1 day before experiments. Apoptosis was induced by treating cells with TRAIL (100 μL, 250 ng/mL) for 2 h at 37° C. The treated apoptotic cells were collected in conical tubes, while the untreated viable cells were trypsinized. The cells were centrifuged at 3000 rpm for 5 mM. The pellets were washed with RPMI 1640 without phenol red. Both apoptotic and viable cells were incubated with 100 μL, of 2 or 3 in RPMI 1640 without phenol red (final concentration, 50 μM) for 2 h at 37° C. The cells were washed and resuspended in annexin-binding buffer (10 mM HEPES, 140 mM NaCl, and 2.5 mM CaCl₂ [pH 7.4]). Five microliters of Alexa Fluor 594-annexin V conjugate was added into each 100 μl of cell suspension, and the cells were incubated for 15 min at room temperature. After washing with PBS, the cells were transferred into Lab-Tek II chambered cover glass (Nalge Nunc International, Naperville, Ill.) and visualized under Olympus Fluoview FV1000 confocal laser scanning microscope (FV1-ASW, Olympus, Melville, N.Y.) equipped with fluorescein isothiocyanate (wavelength: 495/519 nm) and Texas red filters (wavelength: 589/615 nm). For blocking groups in separate wells, TRAIL was added in the presence of 50 μM of a broad-spectrum caspase inhibitor, Ac-DEVD-CHO (Sigma-Aldrich, St. Louis, Mo.).

Cellular uptake and efflux of DEVD peptides 2 and 3—fluorescence analysis DLD1 cells were seeded (1×10⁴/well) in a 96-well plate 1 day before experiments. The DLD1 cells were sequentially treated with 100 μL of the following agents at 37° C.: (A) 25 μM each DEVD conjugate for 2 h; (B) 25 μM each DEVD conjugate for 2 h followed by TRAIL (150 ng/L) for 2 h; (C) 25 μM each DEVD conjugate for 2 h, drug-free culture medium for 2 h, and TRAIL (150 ng/L) for 2 h; and (D) 25 μM each DEVD conjugate for 2 h, drug-free culture medium for 24 h, and TRAIL (150 ng/L) for 2 h. The cells were washed with PBS twice before each medium replacement. After treatment, the cells were lysed with 100 μL of lysis buffer (Sigma) for 15 mM at room temperature. The fluorescence intensity of cleaved R110 was measured using a TECAN microplate reader (San Jose, Calif.). The measurement parameters were as follows: excitation wavelength, 485 nm; emission wavelength, 520 nm; excitation and emission bandwidth, both 12.0 nm; gain, 50; number of flashes, 10; and integration time, 30 μs. The measurements were performed 4 times each.

Cellular uptake and efflux of ^{99m}Tc-chelate 4—radiotracer analysis For cell uptake study, DLD1 cells were grown in 6-cm petri dishes to subconfluent densities in DMEM/F12 culture medium containing 10% fetal bovine serum (FBS) 1 day before experiments. The medium was replaced with 2 mL of medium containing radiotracers 99 mTc-chelate 4 [Ac-DEVD-R110-D-SAAC-99mTc(CO)3] (˜40 μCi/mL) or its fragment R110-D-SAAC-99 mTc(CO)₃ (˜40 μCi/mL). The cells were subsequently incubated for 60 and 120 min before the monolayers were scraped, transferred into 5-mL tubes. The tubes were briefly vortexed and 100 μL DLD1 cell suspension were transferred into a microcentrifuge tube containing 500 μL of a 75:25 mixture of silicon oil (density 1.05, Aldrich) and mineral oil (density 0.872, Acros). The mixture was centrifuged at 14,000 rpm for 5 min. After freezing the tubes with liquid nitrogen, the bottom tips containing the cell pellet were cut off. The cell pellets and the supernatants were counted with a γ-counter (Perkin-Elmer). The protein content in 100 μl cell suspension was quantified in a separate experiment using the Bio-Rad protein assay kit according to the manufacturer's protocol. For efflux study, cells were incubated with each radiotracer for 2 hrs, then the medium was removed and the cells washed with medium twice. The cells were incubated with 2 mL of fresh medium for additional 60 min, 120 min, and 180 min. The radioactivity in the cell pellets and media were counted as described before. Activity ratios of the cell pellet to medium ([cpm/g protein in pellet]/[cpm/g medium]) were calculated and plotted against time. The experiments were performed in pentaplicate.

Fluorescence microscopic analysis of liver from mice injected ^{99m}Tc-chelate 4 Liver tissue was dissected and cryosectioned at 5-μm depth. The tissue slices were fixed using 4% paraformaldehyde for 15 min at room temperature. After PBS washing, the samples were blocked with 10% goat serum at 37° C. for 30 min, followed by 1 μg/mL of rabbit anti-mouse-activated caspase 3 monoclonal antibody (R&D Systems, Minneapolis, Minn.) at 4° C. overnight. Texas red-conjugated goat anti-rabbit IgG (1:1000, Invitrogen, Eugene, Oreg.) was used as second antibody at room temperature for 1 h. Cell nuclei were counterstained with 3 μg/mL of Hoechst 33342 at room temperature for 5 min. The slices were mounted and visualized under a Zeiss Axio Observer.Z1 fluorescence microscope (Carl Zeiss MicroImaging GmbH, Göttingen, Germany). Cleaved ^{99m}Tc-chelate 4 was visualized with an FITC filter, activated caspase 3 with a Texas red filter, and Hoechst 33342 with a UV filter.

LC-MS and HPLC analysis of livers from mice injected ^{99m}Tc-chelate 4 To confirm that the increased radioactivity in the liver of mice treated with anti-Ras antibody was attributed to the retention of fragment R110-SAAC-^{99m}Tc(CO)₃ from cleaved ^{99m}Tc-chelate 4, we analyzed the composition of the extracts from anti-Ras- and PBS-treated liver tissues using radio-HPLC. Two hours after intravenous injection of 4 (1 mCi, 80 μg, 0.2 mL) into a mouse treated with anti-Ras and a mouse treated with PBS 2 h prior to the injection of the radiotracer, mice were killed and livers were removed and ground at 4° C. using a Dounce homogenizer (Polytron, Littan, Switzerland) in 5 mL of acetonitrile/methanol (3:1, v/v). After sequential high-speed centrifugation (100,000 g for 20 min) to remove cellular debris and insoluble proteins, the supernatants were concentrated and injected into the radio-HPLC system (condition: solvent A, 0.01 M NH₄OAc in water; solvent B, acetonitrile; gradient: 0-80% B in A over 40 min, flow rate 1.0 mL/min, C18 4.6×250 mm, 5-μm column). Because Re complexes share similar physical properties to those formed with ^{99m}Tc and are often used as a nonradioactive alternative to ^{99m}Tc for structural characterization, Re-chelate 3 and its corresponding fragment R110-SAAC-Re(CO)₃ were used as references to identify radioactive peaks arising from ^{99m}Tc-chelate 4 and R110-SAAC-^{99m}Tc(CO)₃.

### Example 3

### Cell-Permeable ^{99m}Tc(CO₃)-Labeled Fluorogenic Caspase 3 and 7 Substrate for Dual Modality Detection of Apoptosi

All amino acid derivatives were purchased from Novabiochem (Pasadena, Calif.), Bachem (Torrance, Calif.), and Chem Impex International (Wood Dale, Ill.). Rhodamine 110 (R110) was obtained from Acros (Morris Plains, N.J.). Other chemicals were obtained from Aldrich-Sigma (St Louis, Mo.) and were used as received. Reagent-grade solvents were used without further purification unless otherwise specified. Recombinant human tumor necrosis factor related apoptosis-inducing ligand (TRAIL) was purchased from Millipore (Billerica, Mass.). Alexa Fluor 594-annexin V conjugate, fetal bovine serum (FBS), and RPMI-1640 culture media were purchased from Invitrogen (Carlsbad, Calif.). Caspase-3 and caspase-3 inhibitor Ac-DEVD-CHO were purchased from Aldrich-Sigma.

Liquid chromatography-high resolution mass spectra (LC-HRMS) was performed on an Agilent LC-MSD-TOF system in the positive ion mode using the electrospray ionization method. 1H and 13C NMR spectra were recorded on a Bruker DRX-500 spectrometer (Woodland, Tex.). Preparative RP-HPLC was run on an Agilent 1200 system (C-18, Vydac, 10×250 mm, 10 tim).

Fmoc-protected single amino acid chelate (Fmoc-SAAC-COrH), Fmoc-Lys[di(2-pyridinemethyl)]-COrH, was synthesized according to Levadala et al. Tripeptide Ac-Asp(OBu-t)-Glu(OBu-t)-Val-COOH were synthesized by Fmoc solid phase peptide chemistry using 2-chlorotrityl resin as the solid support. The peptide was cleaved from the resin with dilute trifluoroacetic acid (TFA). The organometallic precursor (NEt₄)₂[Re(CO)₃Br₃] was synthesized according to the literature procedures.

Synthesis of [Asp(OBu-t)]₂-Rhomamine 110 (S1) To a solution of Fmoc-Asp(OBu-t)-CO₂H (1.65 g, 4 mmol) and 1-[3-(dimethylamino)propyl]-3-ethylcarbodiimide hydrochloride (EDC) (0.788 g, 4 mmol) in 5 mL DMF/pyridine (9:1, v/v) was added R110 (200 mg, 0.55 mmol). The reaction mixture was stirred at room temperature under nitrogen protection overnight. The solvent was evaporated under vacuum, and the residue was re-dissolved in ethyl acetate. The organic solvent was washed sequentially with saturated aqueous NaHCO₃ solution (3 times) and brine (3 times), and was dried over anhydrous Na₂SO₄. After removal of ethyl acetate under vacuum, the residue was treated with 20% diethylamine in dimethylformaide (DMF) for 20 min to remove the Fmoc protecting group. The product was purified using column chromatography on silica gel eluted with CHCl₃/MeOH (10:1, v/v) to yielded 350 mg (90%) of 1S as an orange oil. ¹H NMR (MeOD₃): δ 1.48 (s, 18H). 2.90-3.11 (m, 4H), 4.38 (dd, J₁=8.1, Hz, J₂=4.2 Hz, 2H), 6.77 (d, J=8.4 Hz, 2H), 7.22 (m, 3H), 7.44 (m, 1H), 7.70-7.90 (m, 4H), 8.05 (m 1H); FIRMS calcd for C₃₆Ha₄₁N₄O₉ (M+H) 673.2795, found 673.2892. HPLC: t_{R}=16.5 min (solvent A, 0.1% TFA in water; solvent B, acetonitrile; gradient: 0-90% B in A over 30 min, flow rate 1.0 mL/min, C18 4.6×250 mm, 5 μm column).

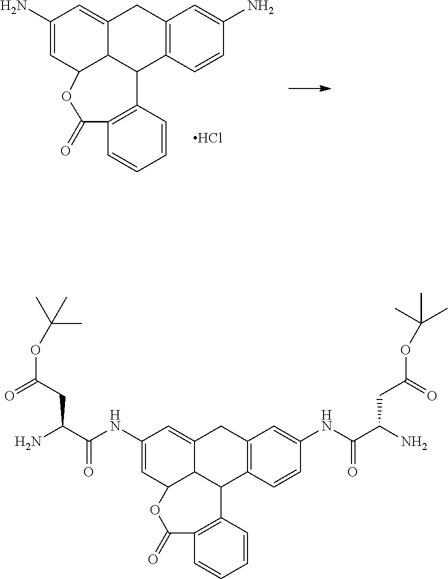

Synthesis of N-Fmoc-SAAC-Asp(OBu-t)-N′-Asp(OBu-t)-Rhodamine 110 (S2) To a solution of Fmoc-SAAC-O₂H (473 mg, 0.86 mmol) and EDC (170 mg, 0.86 mmol) in 2 mL mixture of DMF/pyridine (9:1, v/v) was added S1 (385 mg, 0.57 mmol). The reaction mixture was stirred at room temperature and the progress of the reaction was monitored by LC-MS. Once the peak corresponding to mono-substituted product became maximum, the reaction was stopped by removing the solvent, and the residue was purified by prep HPLC to yield 200 mg (30%) of S2. ¹H NMR (MeOD₃): δ 1.39 (s, 9H), 1.49 (s, 9H), 1.71 (br, 4H), 2.77-3.14 (m, 6H), 3.31-3.36 (m, 6H), 4.09-4.56 (m, 8H), 6.66-6.80 (m, 2H), 7.15-8.07 (m, 22H), 8.66-8.68 (m 2H). HRMS (FAB) calcd for C₆₉H₇₃N₈O₁₂ (M+H) 1205.5270, found (M+H) 1205.5498. HPLC: t_{R}=21.0 min (solvent A, 0.1% TFA in water; solvent B, acetonitrile; gradient: 0-90% B in A over 30 min, flow rate 1.0 mL/min, C18 4.6×250 mm, 5 μm column.

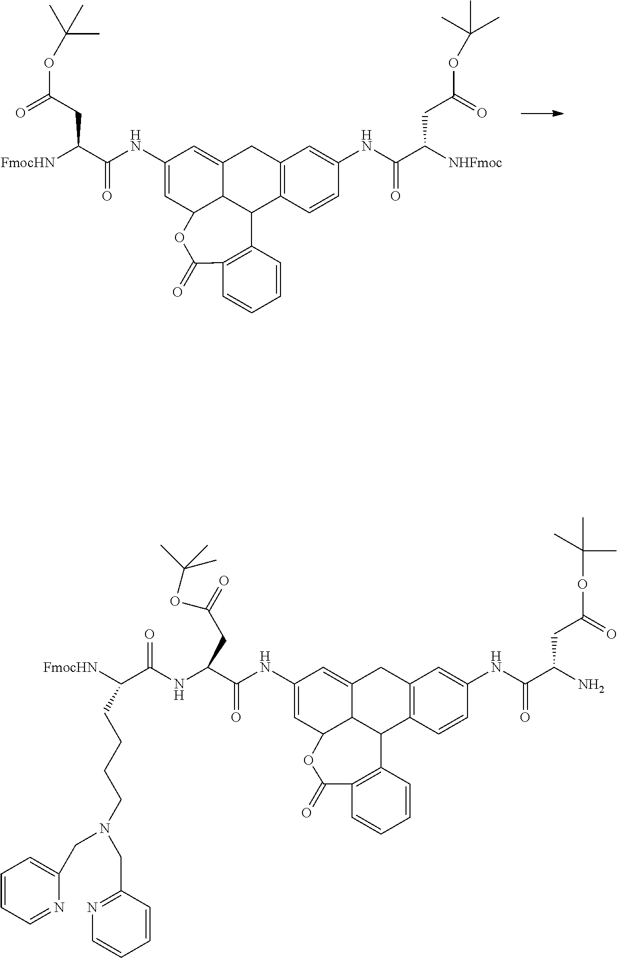

Synthesis of N-(Fmoc-SAAC-Asp)-N′-Asp-Glu-Val-Asp-Rhodamine 110 (2) To the solution of Ac-Asp(OBu-t)-Glu(OBu-t)-Val-CO₂H, (288 mg, 0.56 mmol) in an anhydrous 1:1 mixture of DMF and pyridine (5 mL) was added EDC (110 mg, 0.56 mmol). The solution was stirred at room temperature for 30 min, followed by addition of S2 (116 mg, 0.113 mmol). The solution was stirred at the room temperature for 3 days, then the solvent was removed by vacuum. The residue was dissolved in ethyl acetate and washed with saturated NaHCO₃ (3×), brine (3×), and the organic solution dried over Na₂SO₄. Ethyl acetate was removed under vacuum, and the solid was treated with 50% TFA in dichloromethane (DCM) for 30 min to remove the t-butyl protecting groups. After removal of the solvent, the residue was purified by preparative HPLC to yield 50 mg (30%) of 2. ¹H NMR (MeOD₃): δ 1.00 (m, 2H), 1.45 (br, 2H), 1.65-2.50 (m, 10H), 3.25-3.35 (m, 20H), 4.09-4.62 (m, 10H), 6.60-6.82 (m, 2H), 7.10-8.10 (m, 22H), 8.62-8.64 (m 2H). HRMS calcd for C77H80N₁₁O₂₀ (M+H) 1478.5503, found 1478.5657. HPLC: t_{R}=18.1 min (solvent A, 0.1% TFA in water; solvent B, acetonitrile; gradient: 0-90% B in A over 30 min, flow rate 1.0 mL/min, C18 4.6×250 mm, 5 μm column).

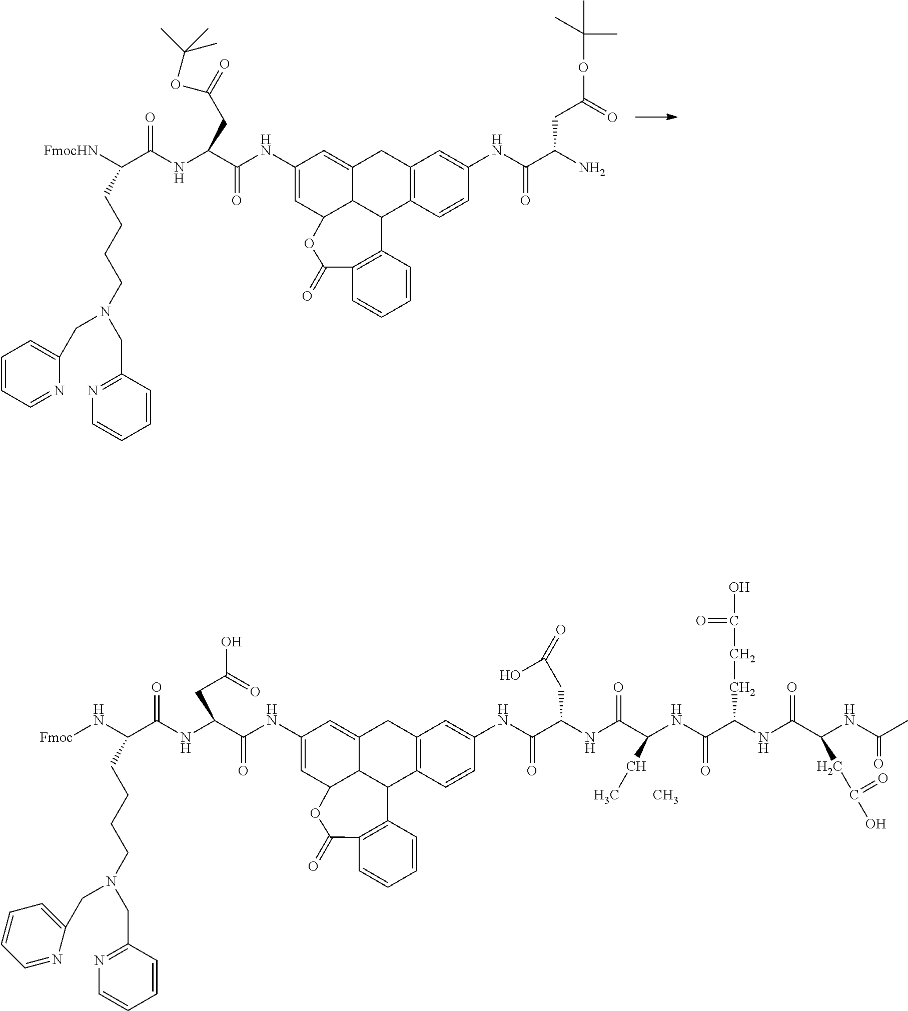

Re-Chelate (3) The Re complex was prepared in quantitative yield by adding a stoichiometric amount of (NEt₄)₂[Re(CO)₃Br₃] in methanol to 3. HRMS calcd for C₈₀H₇₉N₁₁O₂₃Re (m/z) 1748.4908, found 1748.5588. HPLC: t_{R}=20.9 min (solvent A, 0.1% TFA in water; solvent B, acetonitrile; gradient: 0-90% B in A over 30 min, flow rate 1.0 mL/min, C18 4.6×250 mm, 5 μm column). t_{R}=30.9 min (solvent A, 0.1% TFA in water; solvent B, acetonitrile; gradient: 10-50-80% 13 in A over 36 min, flow rate 1.0 mL/min, C18 4.6×250 mm, 5 μm column). The same conditions were used in the analysis of 99 mTc-chelate (see the following section).

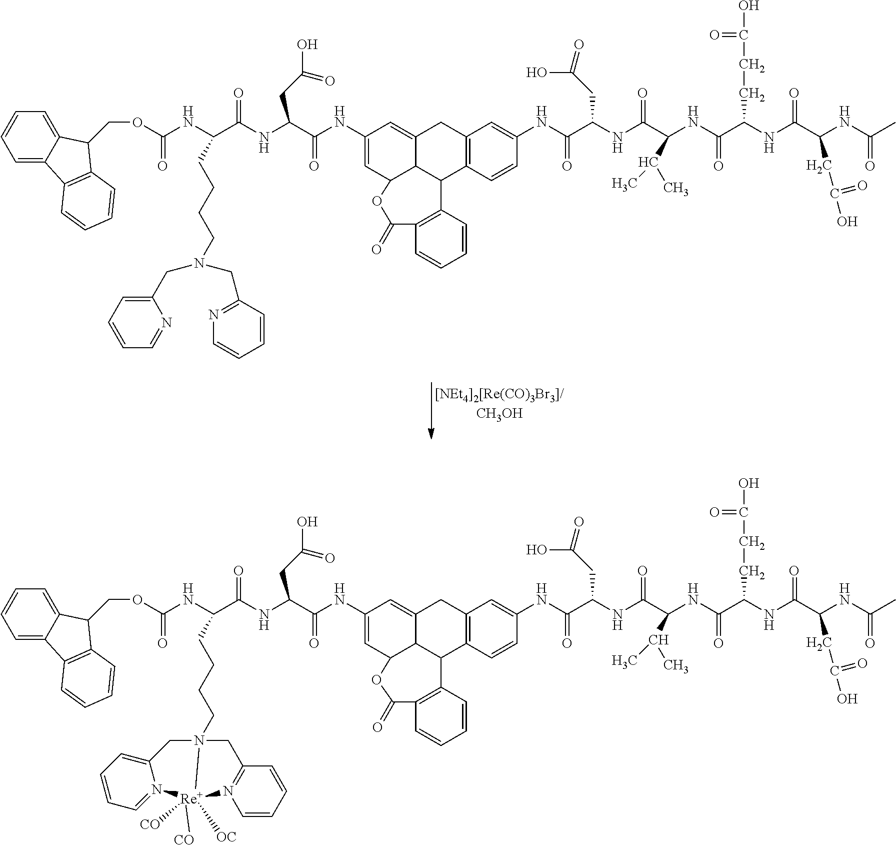

^{99m}Tc Chelate (4) [^{99m}Tc(CO)₃(H₂O)₃]⁺¹ was prepared using the following general procedure: 1.0 mL of ^{99m}TcO₄ (50-200 mCi) was added to commercially available Isolink carbony kits (Mallinckrodt, St. Louis, Mo.). The solution was heated in an oil bath at 100° C. for 20 min. The solution was then cooled for 5 min, vented, and added 120 μL 1N HCl to adjusted pH to 6-7 and to decompose any residual boranocarbonate. In a sealed vial containing 10-20 μg peptide 2 in distilled water was added 0.5 mL ^{99m}Tc—(CO)₃(H₂O)₃ (2 mCi). The vial and heated for 30 min at 75° C. After the sample was cooled, the complexes were characterized using HPLC. The Labeling efficiency was more than 95%. HPLC: t_{R}=31.2 min (solvent A, 0.1% TFA in water; solvent B, acetonitrile; gradient: 10-50-80% B in A over 36 min, flow rate 1.0 mL/min, C18 4.6×250 mm, 5 μm column, UV detector: Agilent 1100 210 set at 254 nm; radioactivity detector: Bioscan flow-count radio-HPLC detector.

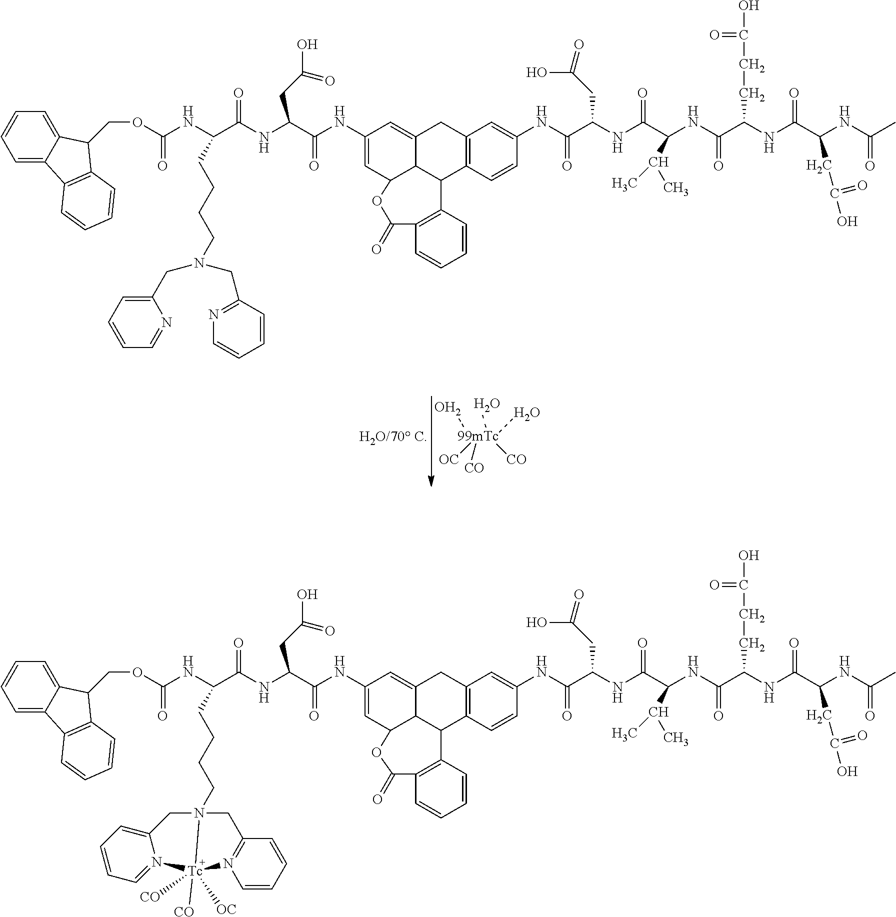

Procedure for biodistribution study All experiments were performed in accordance with the guidelines of the Institutional Animal Care and Use Committee. Female nude mice (20-30 g, Harlan Sprague Dawley, Inc., Indianapolis, Ind.) were divided into 2 groups consisting of 5 mice in each group. Mice were injected ^{99m}Tc-chelate 4 intravenously through the tail vein at a dose of 2 nmol/mouse (5 μCi/mouse). Animals in each group were killed with CO₂ exposure at 5 and 60 min after radiotracer injection. The organs of interest were excised, weighed and the radioactivity counted in a gamma counter. Bladder and excreted urine were not weighed. The stomach and intestines were not emptied of food contents prior to radioactivity measurements. The percentage of injected dose per gram (% ID/g) was calculated by dividing the % ID/organ by the weight of the organ or tissue.

Procedure for cellular uptake Human epithelial cells from colorectal adenocarcinoma (DLD-1) were obtained from American Type Cell Culture (ATCC, Manassas, Va.). Cells were seeded (1×10⁴/well) in 96-well plate (Corning, Lowell, Mass.) supplemented with RPMI-1640 media plus 10% FBS 1 day before experiment. The apoptosis was induced by treating cells with TRAIL (100 μl, 250 ng/ml) for 2 h at 37° C. The treated apoptotic cells were collected in conical tube, while the untreated viable cells were trypsinized. The cells were centrifuged at 3000 rpm for 5 min. The pellets were washed with RPMI-1640 without phenol red. Both apoptotic and viable cells were incubated with 100 μl of 2 or 3 in RPMI-1640 without phenol red (final concentration, 50 μM) for 2 h at 37° C. The cells were washed and resuspended in annexin-binding buffer (10 mM HEPES, 140 mM NaCl, and 2.5 mM CaCl₂, pH 7.4). Five microliters of Alexa Fluor 594-annexin V conjugate was added into each 100 μL of cell suspension, and the cells incubated for 15 min at room temperature. After washing with PBS, the cells were transferred into Lab-Tek II Chambered cover glass (Nalge Nunc International, Naperville, Ill.) and visualized under Olympus IX81 confocal microscope (Olympus, Japan) equipped with FITC (wavelength: 495/519 nm) and Texas Red filters (wavelength: 589/615 nm). Differential interference contrast (DIC) images were taken under bright field at the same visual area. For blocking group in a separate well, TRAIL was added in the presence of 50 μM of a broad-spectrum caspase inhibitor, Ac-DEVD-CHO.

Uptake and release kinetics of DEVD peptides in normal and apoptotic cells. DLD-1 cells were seeded (1×10⁴/well) in 96 well plate 1 days before experiment. The DLD1 cells were sequentially treated with 100 μL of the following agents at 37° C.: (A) 25 μM of each DEVD conjugate for 2 h followed by drug-free culture media for 2; (B) 25 μM of each DEVD conjugate for 2 h followed by TRAIL (150 ng/L) for 2 h; (C) 25 μM of each DEVD conjugate for 2 h, drug-free culture media for 2 h, and TRAIL (150 ng/L) for 2 h; (D) 25 μM of each DEVD conjugate for 2 h, drug-free culture media for 24 h, and TRAIL (150 ng/L) for 2 h. The cells were washed with PBS twice before each medium replacement. After treatment, the cells were lysed with 100 μL of lysis buffer (Sigma) for 15 min at room temperature. The fluorescent intensity of cleaved R110 was measured using TECAN microplate reader (San Jose, Calif.). The measurement parameters are listed as follows: excitation wavelength 485 nm, emission wavelength 520 nm, excitation and emission bandwidth both 12.0 nm, gain 50, number of flashes 10, integration time 30 μs. The measurements were performed in tetraplicate.

In summary, the novel cell-permeable imaging probes taught herein are suitable for both fluorescence microscopy and nuclear imaging of caspase 3 activity in apoptotic cells. Both in vitro and in vivo data support our hypothesis that the underlying mechanism for satisfactory nuclear imaging is attributable to reversible diffusion of the parent substrate in viable cells and increased retention of the radioactive fragment cleaved by activated caspase 3 in apoptotic cells. The combined use of two powerful molecular imaging methods provides the opportunity for a direct correlation between in vitro and in vivo biological activities, and allows validation of nuclear imaging using ex vivo fluorescence microscopy technique.

Methods of dual optical and nuclear imaging of enzymatic activity using the single imaging probes as taught herein are useful for defining the pharmacokinetics, optimal imaging protocol, and the suitability of 4 for non-invasive detection of apoptosis in various disease models, including apoptotic response of solid tumors to anticancer therapy. Fluorogenic dyes that emits fluorescent signal in the near-infrared region upon activation are also needed for in vivo optical imaging applications because of the deep tissue penetration of near-infrared light.

We claim:

1. A conjugate compound comprising a caspase peptide substrate, DEVD, and a fluorogenic compound and a radioactive tag wherein said conjugate is activated upon cleavage from DEVD in apoptotic cells.

2. A conjugate compound for use in dual optical and nuclear imaging of enzymatic activity or the treatment of disease comprising DEVD-R110-SAAC, or a Re or ^{99m}Tc chelate thereof.

3. A single imaging probe comprising the conjugate of claim 1.

4. A method of dual optical and nuclear imaging of enzymatic activity using the single imaging probe comprising the steps of exposing tumor cells to the conjugate compound of claim 1 wherein a fluorescent signal is produced.
